(19) United States
(12) Patent Application Publication
(10) Pub. No.: US 2025/0388953 A1
(43) Pub. Date: Dec. 25, 2025

(54) MULTIPLEXABLE APTAMER-BASED LIGAND DETECTION

(71) Applicant: THE GOVERNING COUNCIL OF THE UNIVERSITY OF TORONTO, Toronto (CA)

(72) Inventors: Andrew G. Fraser, Toronto (CA); June H. Tan, Scarborough (CN); Maria P. Mercado, Whitby (CA)

(21) Appl. No.: 18/706,491

(22) PCT Filed: Nov. 2, 2022
(86) PCT No.: PCT/CA2022/051618

§ 371 Date: May 1, 2024

(30) Foreign Application Priority Data
Nov. 2, 2021 (SE) 2151343-7

(51) Int. Cl. C12Q 1/6823; C12N 15/115; C12Q 1/6834; C40B 20/04; C40B 30/04; C40B 40/06

(52) U.S. Cl. CPC C12Q 1/6823; C12N 15/115; C12Q 1/6834; C40B 20/04; C40B 30/04; C40B 40/06; C12N 2310/16; C12N 2320/10; C12Q 2600/16

(57) ABSTRACT

Described herein are multiplexable aptamer-based systems and methods for detecting target ligands in a fluid sample. More specifically, described herein are ligand-sensing complexes comprising a ligand-binding oligonucleotide (LBO) hybridized to a corresponding short-release oligonucleotide (SRO) such that binding of a target ligand to the LBO drives a conformational change triggering release of a barcoded SRO or LBO. The released barcode, which comprises a sequence that is informative with respect to the target ligand bound, may then be captured, amplified and/or sequenced as a readout for the presence/concentration of the target ligand in the fluid sample. Also described herein is a method for preparing ligand-sensing complexes with error-free LBO/SRO pairing, as well as a method for improving the sensitivity and/or dynamic range of aptamer-based detection systems.

The present description relates to multiplexable aptamer-based detection of ligands in a fluid sample. More specifically, the present description relates to ligand-sensing complexes such as based on structure-switching aptamers that release informative barcode sequences upon ligand binding, the released barcode sequences being useful as a readout for the presence/concentration of the target ligand in the fluid sample. The present description also relates to aptamer-based detection systems having improved sensitivity and/or dynamic range for a target ligand.

The present description refers to a number of documents, the contents of which are herein incorporated by reference in their entirety.

### BACKGROUND

Biological fluids such as tears, plasma, or urine, contain complex mixtures of small molecules that include but is not limited to endogenous metabolites and exogenous drugs. Measuring these efficiently is powerful for diagnostics in many fields. Currently almost all measurements of small molecules require mass spectrometry or high-performance liquid chromatography (HPLC) and thus needs expensive machines, diagnostics labs, and relatively large sample volumes. Structure switching aptamers (SSAs) provide an alternative—each SSA binds a specific small molecule ligand and ligand binding induces a conformational change that can be detected through fluorescence or conductance. These readouts have a major limitation, however, since every SSA has the same readout, and it is thus impossible to multiplex many hundreds or thousands of such SSAs in a single assay volume. In addition, it is not possible to amplify the signal. These two factors make the use of SSAs in situations where either analyte volume is limiting or where the small molecule ligands are at very low abundance—such as in single cell metabolomics—very difficult since sample volumes and metabolite levels are very low. There is therefore a need for new and streamlined multiplexed systems for detecting analytes using SSAs.

### SUMMARY

In a first aspect, described herein is a system for detecting one or more target ligands in a fluid sample, the system comprising one or more ligand-sensing complexes, each ligand-sensing complex being specific for a target ligand and comprising a ligand-binding oligonucleotide (LBO) hybridized to a corresponding short-release oligonucleotide (SRO) to form an LBO/SRO pairing, the LBO comprising a ligand-binding region that specifically binds to the target ligand and an SRO hybridization region sufficiently complementary to a corresponding LBO hybridization region comprised in the SRO to enable formation of the LBO/SRO pairing, wherein the SRO or LBO further comprises a barcode region informative with respect to the target ligand recognized by the ligand-sensing complex, and wherein binding of the target ligand to the ligand-binding region of the LBO drives a conformational change triggering release of the barcoded SRO or LBO the LBO/SRO pairing, thereby enabling the released barcode region to be used as a readout for the presence or concentration of the target ligand in the fluid sample.

In a further aspect, described herein is a method for detecting or measuring a ligand in a fluid sample, the method comprising:
(a) providing the system described herein;
(b) contacting the system with the fluid sample;
(c) detecting the released barcoded SRO(s) as a readout for the presence or concentration of each of the target ligand(s) in the fluid sample.

In a further aspect, described herein is a method for preparing one or more ligand-sensing complexes described herein, the method comprising:
(a) providing one or more unimolecular polynucleotides, each unimolecular polynucleotide comprising the nucleotide sequences of both the LBO and the SRO separated by a cleavage site (e.g., at an abasic site, a photocleavable site, or an enzymatic cleavage site) positioned therebetween;
(b) allowing the complementary LBO and SRO hybridization regions to hybridize; and
(c) cleaving the polynucleotide at the cleavage site, thereby producing the one or more ligand-sensing complexes having separate LBO and SRO molecules that are correctly paired.

In a further aspect, described herein is a unimolecular polynucleotide described herein, preferably for use in preparing a ligand-sensing complex described herein.

In a further aspect, described herein is an aptamer-based detection system having improved (e.g., expanded or broadened) dynamic range for a target ligand, the system comprising a plurality of ligand-binding oligonucleotide (LBO) species that bind to the same target ligand. In some embodiments, the plurality of LBO species comprises LBO species having different ligand-binding regions that specifically bind to the target ligand but with different affinities. In some embodiments, the plurality of LBO species comprises LBO species having identical ligand-binding regions that specifically bind to the target ligand but that differ in their non-ligand binding structural elements. In some embodiments, the plurality of LBO species comprises both: (a) LBO species having different ligand-binding regions that specifically bind to the target ligand but with different affinities, and (b) LBO species having identical ligand-binding regions that specifically bind to the target ligand but that differ in their non-ligand binding structural elements. The resulting aptamer-based detection system thus comprises a plurality LBO species that bind to the same target ligand at different but overlapping dynamic ranges such that the overall dynamic range of the system is greater than that of a single LBO species.

In a further aspect, described herein is a method for increasing the sensitivity and/or dynamic range of an aptamer for its ligand, the method comprising: providing a sample comprising or suspected of comprising a ligand of interest; contacting the sample with an aptamer that binds to the ligand of interest in the presence of a concentration of an inert macromolecular crowding agent sufficient to increase the aptamer's sensitivity and/or dynamic range with respect to its ligand, as compared to the aptamer's sensitivity and/or dynamic range in a corresponding sample lacking the inert macromolecular crowding agent.

### BRIEF DESCRIPTION OF THE DRAWINGS

In the appended drawings:

FIGS. 1A to 1D relate to methods to generate libraries of barcoded structure-switching aptamers. FIG. 1A: Each barcoded sensor is comprised of two parts—a ligand binding oligo (LBO) and a short-release oligo (SRO). When the LBO is bound by its cognate ligand, a conformation change induces the release of an SRO that contains a DNA barcode sequence specific to that sensor, which can then be amplified and sequenced. FIG. 1B: Each two-part SSA can be assembled separately by base-pairing via the same universal sequence. LBOs and SROs are paired separately for each SSA and pooled together at a later stage to generate the SSA library. FIG. 1C: SSAs can be assembled in parallel by utilizing unique base-pairing sequences. FIG. 1D: SSAs can be synthesized first as unimolecular pre-SSAs that contain both the LBO and barcoded SRO regions. Each pre-SSA contains a specific site (such as an abasic site or a restriction enzyme site for a nicking restriction enzyme) preceding the common base-paired SRO-LBO region, which can be used to direct a nicking cleavage to convert the unimolecular pre-SSA to the mature bimolecular SSA.

FIGS. 2A to 2C relate to methods for detecting ligand binding. FIG. 2A Method for detecting ligand binding by Yang et al., 2014 and other groups. An LBO base-pairs with an SRO bound to an agarose-streptavidin column or other solid matrices. Ligand binding to the LBO induces a conformational change that releases the entire LBO from the solid matrix. FIG. 2B Method for detecting ligand binding using barcode SSA. Here, we instead have the LBO attached to a solid matrix (streptavidin magnetic beads or other solid matrices) while the SRO is released upon ligand binding. FIG. 2C Ligand binding to the LBO results in release of the corresponding barcode SRO, which can then be amplified and sequenced. This method is highly scalable and can be used to detect ligand binding in parallel for a large library of SSAs since each SSA is associated with a unique ligand binding specificity and releases a unique known barcode. Individual, tens, hundreds, thousands, or millions of barcodes can be independently detected in parallel at any one time.

FIGS. 3A to 3E relate to the readout of barcode SRO release using a fluorescent reporter. FIG. 3A: Schematic of SSAs used in proof-of-concept experiments. To detect amounts of released barcode SRO, we used a T7 promoter sequence in place of the barcode sequence. This T7 promoter sequence can then be added to a transcription template to allow transcription of any reporter sequence. FIG. 3B: Schematic of T7-Spinach fluorescence assay. To detect SRO release using a fluorescent reporter, the SRO sequence containing a T7 promoter is annealed to a transcription template for Baby Spinach. The Baby Spinach RNA is then transcribed by T7 RNA polymerase in an in vitro transcription reaction. DFHBI fluoresces only when bound to the folded Baby Spinach aptamer, allowing detection of the transcribed products. FIG. 3C: Various amounts of T7 primers were titrated into a reaction containing 20 pmol of the transcription template. After a 2 h in vitro transcription reaction, the transcribed products were heated at 90° C. for 2 min, then placed on ice for 5 min. DFHBI was then added at a final concentration of 10 μM, and the mixture was then heated at 65° C. for 5 min and slowly cooled to room temperature. Fluorescence intensity for each sample was then measured using a FLUOstar™ Omega microplate reader (excitation 485 nm, emission 520 nm). FIG. 3D: Image of the fluorescence samples shown in FIG. 3C. FIG. 3E: A series of 2-fold dilutions of T7 barcode SROs added to 20 pmol of Baby Spinach transcription template. Samples were then transcribed, DFHBI-1T-treated and fluorescence was measured as described above.

FIGS. 4A to 4J relates to a ligand binding assay using a T7 barcode SRO. FIG. 4A: Outline of ligand binding assay. A biotinylated LBO is hybridized to a barcode SRO that contains a T7 promoter sequence. The paired SRO-LBO duplex is then bound to streptavidin-coated MyOne™ C1 Dynabeads™ Amino acids are then complexed to Cp*Rh(III) and added to the bound SSAs. Upon binding of the target complex, the T7 SRO barcode dissociates from the LBO and is released into the supernatant. This released fraction is then added to the downstream transcription reaction to measure SRO release. FIG. 4B: Percent SRO released in response to Trp using an LBO sequence that corresponds to the Trp aptamer described in Yang et al., 2014. The supernatant was collected after a 45 min incubation with ligand (supernatant fraction). The beads were then resuspended and boiled for 4 min to release all remaining SRO (bound fraction). Percent release represents the fluorescence intensity detected in the supernatant fraction relative to the fluorescence intensity detected in both the supernatant fraction and the remaining bound fraction. Specific SRO release is observed in response to 10 μM Trp and not Leu or Phe addition. FIG. 4C: SRO released upon ligand addition using an LBO sequence that corresponds to the Phe or Tyr aptamer described in Yang et al., 2014. Cp*Rh(III) in buffer alone was used as the negative control. FIG. 4D: Specificity of the Phe and Tyr SSAs in response to 10 μM ligand. FIG. 4E: Minimal SRO is released upon 100 μM Phe addition when using a scrambled LBO sequence. FIG. 4F: Dose response of Phe and Tyr SSAs to their cognate ligands. A four-parameter logistic (4 PL) model was used for curve fitting. FIG. 4G: SRO released in response to piperaquine using an LBO sequence that corresponds to the PQ4 aptamer, as described in Coonahan et al., 2021. FIG. 4H: SRO released upon mefloquine addition using an LBO sequence that corresponds to the MQ2 aptamer, as described in Coonahan et al., 2021. FIG. 4I: SRO released upon cortisol addition using cortisol LBO. FIG. 4J: SRO released upon ampicillin addition using ampicillin LBO.

FIGS. 5A to 5D relate to the ligand binding assay in a mixture of SSAs. FIG. 5A: The Phe LBO is paired to a T7 barcode SRO while the LBO with the scrambled ligand-binding region is paired to a T3 barcode SRO. The two SSAs were mixed in equal amounts then subjected to the same ligand binding assay described above. Released SROs were added to a T7 Baby Spinach transcription template and the template was transcribed. Fluorescence intensity was then measured in each sample after addition of DFHBI-1T. Upon addition of 100 μM Phe, fluorescence corresponding to T7 barcode is observed, while minimal fluorescence is observed in the absence of Phe. FIG. 5B: The Phe LBO is paired to a T3 barcode SRO while the LBO with the scrambled ligand-binding region is paired to a T7 barcode SRO. SSAs were mixed and assayed as above. Minimal fluorescence is observed with or without Phe addition. FIG. 5C: The piperaquine (PQ) LBO is paired to a T7 barcode SRO while the mefloquine (MQ) LBO is paired to a T3 barcode SRO. The two SSAs were mixed in equal amounts then incubated with either 50 μM PQ or MQ. Error bars represent standard deviation. FIG. 5D: The PQ LBO is paired to a T3 barcode SRO while the MQ LBO is paired to a T7 barcode SRO. SSAs were mixed and assayed as described above.

FIGS. 6A to 6C relate to the multiplexing of SSAs using different fluorophores. FIG. 6A: A fluorescence readout where target binding induces a conformational change that results in displacement of a short oligo attached to a fluorophore. The fluorescence intensity of the released fraction is then directly measured to determine the amount of SRO released. A piperaquine (PQ)-binding LBO is paired to a 6-FAM SRO, while a cortisol-binding LBO is paired to a TEX615 SRO. The two SSAs were mixed in equal amounts then incubated with either 50 μM of PQ (FIG. 6B) or 200 μM of cortisol (FIG. 6C). Fluorescence intensity was then measured in each sample in two different channels.

FIGS. 7A to 7F relate to converting previously identified aptamers to our barcode SSA platform. Schematics of an LBO in which the ligand-binding region is replaced by the ampicillin-binding AMP17 aptamer sequence and hybridized to an SRO conjugated to a fluorophore (FIG. 7A) or to a DNA barcode in the form of a T7 promoter sequence (FIG. 7C). FIG. 7B: Percent release of fluorescently labeled SRO after ampicillin addition. FIG. 7D: Percent release of T7 SRO after ampicillin addition. The dose response curve for the Amp SSA was fitted using a 4 PL logistic model. FIG. 7E: Specificity of the Amp SSA in response to 500 μM Amp, carbenicillin (Carb) or kanamycin (Kan). FIG. 7F: Percent release of SRO after addition of LB+ampicillin mixture. Various amounts of ampicillin were added to LB broth, and SSAs were then incubated in these mixtures. To maintain similar salt conditions as used for previous SSA experiments, NaCl, MgCl₂ and KCl were added to the LB medium at a final concentration of 1 M, 10 mM and 5 mM respectively. LB medium with no ampicillin added was used as the control, and the difference in SRO released with Amp relative to no Amp is plotted.

FIGS. 8A to 8D relate to the use of SSAs in complex mixtures. FIG. 8A: SRO release in LB spiked with various amounts of piperaquine (PQ). LB medium with no PQ added was used as the control, and the difference in SRO released with PQ relative to no PQ is plotted. FIG. 8B: SRO release in Caenorhabditis elegans worm lysate spiked with PQ. Lysate with no PQ added was used as the control, and the difference in SRO released with PQ relative to no PQ is plotted. FIG. 8C: Drug detection in C. elegans worms treated with piperaquine. bus-5(br19) worms were treated with either piperaquine or no drug for 7 h. Worms were then lysed, and PQ SSAs were incubated in worm lysate to monitor SRO release. The mean of 2 biological replicates is plotted with error bars representing standard deviation. FIG. 8D: Specific detection of piperaquine and mefloquine in drug-treated worms. bus-5(br19) worms were treated with either piperaquine, mefloquine or no drug for 7 h. Worms were then lysed and either PQ (left) or MQ (right) SSAs were incubated in the different drug-treated worm lysates to monitor SRO release.

FIG. 9 relates to the use of different hybridization sequences with similar SRO release parameters. Dose response curves of Amp SSAs using sensors with scrambled hybridization sequences. Here, the LBO/SRO base-pairing region in the Amp SSA was scrambled (dotted box).

FIGS. 10A to 10D relate to SSAs that can be assembled by cleaving a unimolecular pre-SSA to generate functional, paired SRO and LBO sequences. FIG. 10A: Schematic of SSA library assembly. A library of barcoded SSAs can be generated by first synthesizing a pre-SSA oligo that contains both the LBO and SRO sequences. These pre-SSA sequences each contain an abasic site that can be cleaved to generate the bimolecular mature SSA construct. These pre-SSAs are then folded so that the LBO and SRO sequences are base-paired. The folded constructs are then cleaved at the abasic site using Endonuclease IV, generating mature SSAs. This pre-SSA assembly method is used to generate the same Trp SSA sequence as described in FIG. 4B. FIGS. 10B and 10C: Percent SRO release after 10 μM Trp addition. With Trp addition, the mature Trp SSA shows SRO release as expected while the aptamer with a scrambled ligand-binding region shows minimal SRO release. FIG. 10D: SRO is released in a dose-dependent manner in response to Trp.

FIGS. 11A and 11B relate to increasing the chemical and physical space of the ligand binding region. FIG. 11A: To reduce the complexity of barcode SSA libraries, we vary a maximum of 12 possible positions within the ligand binding region of the LBO. The most basic form is shown at the top: a linear sequence of 12 bases. To increase the physical dimension of the ligand binding region, we can insert constant regions into that N12 variable sequence. Here we show 3 architectures: SINGLE insertion regions, DOUBLE insertion regions or TRIPLE insertion regions. The insertions can be fixed nucleotides (e.g., C1 denotes a single fixed nucleotide, C6 denotes 6 fixed nucleotides, and these can be RNA, DNA or XNA) or inert spacers such as a 3 or 6 length polyethyleneglycol spacer. In the diagram, GREY regions are variable regions, BLACK regions are constant sequence regions and LIGHT GREY denotes other spacer chemistries. LIGHT GREY is only shown in the SINGLE spacer example, but BLACK can be replaced with LIGHTGREY throughout. FIG. 11B: SRO released in response to tyrosine using an LBO sequence that corresponds to the tyrosine aptamer (‘Tyr’) as described in Yang et al., 2014. 7 bases in the ligand-binding region of the Tyr aptamer were also replaced with either a sequences of 9 adenines (‘Tyr 9A’) or 9 thymines (‘Tyr 9T’). The aptamers with the 9 fixed As or Ts both respond to 200 μM tyrosine in a similar manner as the original Tyr aptamer.

FIGS. 12A to 12C relate to the use of a T7 promoter to drive transcription and amplification of barcodes. FIG. 12A shows the schematic of an example barcode SRO that is under control of a T7 promoter. A short sequence immediately downstream of the promoter and upstream of the 12 nt barcode is added for increased transcription efficiency. FIG. 12B: 20 pmol of T7 primer is added in excess to 0 or 2 pmol of template (barcode SRO) and annealed to form a double-stranded promoter region. In vitro transcription is then carried out and the transcribed products were visualized on a 4% agarose gel. FIG. 12C: The Phe LBO was paired with the barcode SRO described in (A) and incubated with 0 or 100 μM Phe and released SROs were monitored as in FIG. 12B.

FIGS. 13A to 13C relate to the use of nicking endonucleases to cleave pre-SSAs into LBOs and SROs. FIG. 13A: Nb.BstI only cleaves one strand of dsDNA, and by incorporating its restriction site into the stem region of our SSAs, we could potentially induce a nick at the intersection of the LBO and SRO sequences (ideal cleavage site represented by a yellow star). FIG. 13B: The Nb.BstI restriction site is incorporated into the stem sequence of a PQ SSA and SRO release is measured after incubation with piperaquine. FIG. 13C: Nb.BstI treatment results in cleaved fragments of expected sizes. Lanes 1-5 were samples that were incubated with Nb.BstI for various amounts of time (1-2 h) with various folding conditions and lane 6 was the input control that was incubated without any enzyme. After enzyme incubation, samples were run on a 10% TBE-Urea polyacrylamide gel and stained for 30 min with SYBR™ Gold.

FIGS. 14A and 14B relate to the use of 2′-OMe-RNA/DNA chimeras as LBOs. FIG. 14A: Schematic of PQ LBOs with DNA bases replaced by 2′-OMe-RNA bases in the common stem region (dotted region). Ligand binding was assayed as described in FIG. 3B and the dose response curve plotted on the right. FIG. 14B: Schematic of PQ LBOs with DNA bases substituted by 2′-OMe-RNA bases in 4 nt of the ligand binding region (dotted region). These bases are predicted by mFold to be base-paired and were chosen to minimize likelihood of disrupting specific ligand contacts. The corresponding dose response is plotted on the right.

FIGS. 15A and 15B relate to selections using N12 controlled complexity libraries. FIG. 15A: For aptamer selection experiments, we incorporate an N12 variable region in the ligand-binding region of the LBO. In addition to the basic N12 form (left-most configuration), we also screen additional libraries where the variable bases are surrounded by different fixed and/or structured elements. These can be short regions of fixed nucleotide sequences or insertions of other chemistries such as polyethylene glycol spacers or insertions of fixed secondary structures such as hairpin loops. FIG. 15B: Sequences enriched in our ampicillin selection experiments, which include both N12 sequences as well as sequences with fixed T4 spacers. The highlighted sequences were incorporated into the ligand-binding region of our standard barcode SSA configuration, and the dose response was measured as previously described.

FIGS. 16A to 16D relate to varying sequence composition of stems allowing tuning of response curves. FIG. 16A: Dose response curve of the ‘parent’ piperaquine SSA using a T7 SRO. FIG. 16B: Dose response curve of the ‘parent’ phenylalanine SSA using a T7 SRO readout. FIG. 16C: Dose response curve of variants of the parent piperaquine SSA, where the stem sequences were scrambled and modified but the % GC content kept the same. FIG. 16D: Dose response curve of variants of the parent phenylalanine SSA, where the stem sequences were scrambled and modified but the % GC content kept the same. The specific sequences of the variants are listed in Table 3.

FIGS. 17A and 17B relates to parallel use of multiple sensors for the same ligand to greatly expand dynamic range. The dynamic range is estimated as the linear range of response between 20% and 80% oligo release and illustrated for the piperaquine (FIG. 17A) and phenylalanine (FIG. 17B) SSAs. The values for the parent and variant SSAs are extracted from the dose response curves in FIG. 16C and FIG. 16D. ‘Range’ refers to the combined ranges of the 3 SSA variations. For the parent and combined ranges, the fold change between the maximal and minimal values in their respective ranges are listed. Additional values are listed in Table 4.

FIGS. 18A to 18E relate to the addition of macromolecular crowding reagents for increasing the sensitivity of some SSAs. FIG. 18A: Addition of various concentrations of PEG 8000 increases the sensitivity of the PQ SSA at low PQ concentration. Dose response curves of the piperaquine (PQ) SSA (FIG. 18B) and mefloquine SSA (FIG. 18C) in the presence or absence of PEG 8000 using a direct fluorescence readout. FIG. 18D: Addition of various concentrations of Ficoll™-70 increases the sensitivity of the PQ SSA at low PQ concentrations. FIG. 18E: Dose response curves of the PQ SSA in the presence or absence of Ficoll-70 using a direct fluorescence readout.

FIGS. 19A to 19C relate to barcoded SSAs with an immobilized short common oligo and a released LBO. SSAs could also be configured such that the short common region of the stem is immobilized while the LBO is released upon ligand binding (FIG. 19A). A T7 promoter is attached to the 3′ end of the LBO and LBO sequences released upon ligand binding are then used to transcribe a Baby Spinach template (FIG. 19B). A tyrosine T7 LBO is released and detected in our assay upon addition of tyrosine (FIG. 19C).

FIGS. 20A to 20C show different methods to separate a barcode from rest of the LBO in the architectures as described in FIGS. 19A and 19B. FIG. 20A: An abasic site could be cleaved to separate the barcodes from the rest of the LBO. A common region can also be added either upstream or downstream of the barcode for use in affinity purification. The barcodes can then be pulled down by immobilized anti-sense oligos to purify the barcodes. We note that the barcodes can be isolated in other ways, such as size selection, depletion of the remaining LBO, and other standard methods. FIG. 20B: A restriction site could also be used in lieu of an abasic site, where a short anti-sense oligo could be added to create a double stranded region at the restriction site to enable enzyme binding and cleavage. FIG. 20C: As with the barcode SROs we previously described an antisense version of the barcode and a T7 promoter can be placed upstream of the ligand binding region of the LBO. T7 primers could then be added to transcribe and amplify the barcode region. In this way, only the regions consisting of DNA bases need to be read. The amplified barcodes can then be purified using the methods described above.

### SEQUENCE LISTING

This application contains a Sequence Listing in computer readable form created Nov. 1, 2022. The computer readable form is incorporated herein by reference.

TABLE 1
Sequence Listing Description

| SEQ ID NO: | Description |
|---|---|
| 1 | T7 Baby Spinach transcription template |
| 2 | Phenylalanine (Phe) ligand-binding oligonucleotide (LBO) |
| 3 | Scrambled Phe LBO |
| 4 | Tyrosine LBO |
| 5 | T7 short release oligonucleotide (SRO) |
| 6 | T3 SRO |
| 7 | Piperaquine LBO |
| 8 | Piperaquine T7 SRO |
| 9 | Piperaquine T3 SRO |
| 10 | Mefloquine LBO |
| 11 | Mefloquine T7 SRO |
| 12 | Mefloquine T3 SRO |
| 13 | Ampicillin LBO |
| 14 | Ampicillin Scr1 LBO |
| 15 | Ampicillin Scr2 LBO |
| 16 | Ampicillin Scr1 T7 SRO |
| 17 | Ampicillin Scr2 T7 SRO |
| 18 | Tryptophan structure-switching aptamer (SSA) |
| 19 | Scrambled Trp SSA |
| 20 | Tyrosine (Tyr) 9A LBO |
| 21 | Tyr 9T LBO |
| 22 | Transcribed T7 Baby Spinach template |
| 23 | Exemplary Barcode SRO |
| 24 | T7 Primer |
| 25 | Nb. BstI Exemplary Cleavage Site |
| 26 | Nb. BstI Exemplary Cleavage Site (complementary strand) |
| 27 | Group A N12 Spacer 1 |
| 28 | Group A N12 Spacer 2 |
| 29 | Group A N12 Spacer 3 |
| 30 | Group E N12 Spacer 1 |
| 31 | Group E N12 Spacer 2 |
| 32 | Group E N12 Spacer 3 |
| 33 | Group E N12 Spacer 4 |
| 34 | Group G N12 Spacer 1 |
| 35 | Group G N12 Spacer 2 |
| 36 | Phenylalanine LBO Variant 1 |
| 37 | Phenylalanine LBO Variant 2 |
| 38 | Piperaquine LBO Parent |
| 39 | Piperaquine LBO Variant 1 |
| 40 | Piperaquine LBO Variant 2 |
| 41 | Cortisol LBO |
| 42 | 6-FAM Piperaquine SRO |
| 43 | TEX615 Cortisol SRO |
| 44 | Nb. BstI Piperaquine LBO |
| 45 | Nb. BstI Piperaquine SRO |
| 46 | Ampicillin A4 LBO |
| 47 | Ampicillin E3 LBO |
| 48 | Ampicillin G2 LBO |
| 49 | Ampicillin A4/E3/G2 6-FAM SRO |

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, advantages and features of the present description will become more apparent upon reading of the following non-restrictive description of specific embodiments thereof, given by way of example only with reference to the accompanying drawings.

### DETAILED DESCRIPTION

The present description relates to a readily multiplexable system for detecting target ligands in a fluid sample. The system employs aptamer-based ligand-sensing complexes that release informative barcode sequences upon ligand binding. The released barcode sequences are then used as a readout for the presence/concentration of the target ligands in the fluid sample. Methods for the efficient and error-free assembly of large libraries of the ligand-sensing complexes are also described herein. The present description also relates to aptamer-based detection systems having improved sensitivity and/or dynamic range for a target ligand, for example, by employing multiple ligand-sensing complexes that recognize the same ligand with different binding properties, and/or by the inclusion of an inert macromolecular crowding agent in the fluid sample.

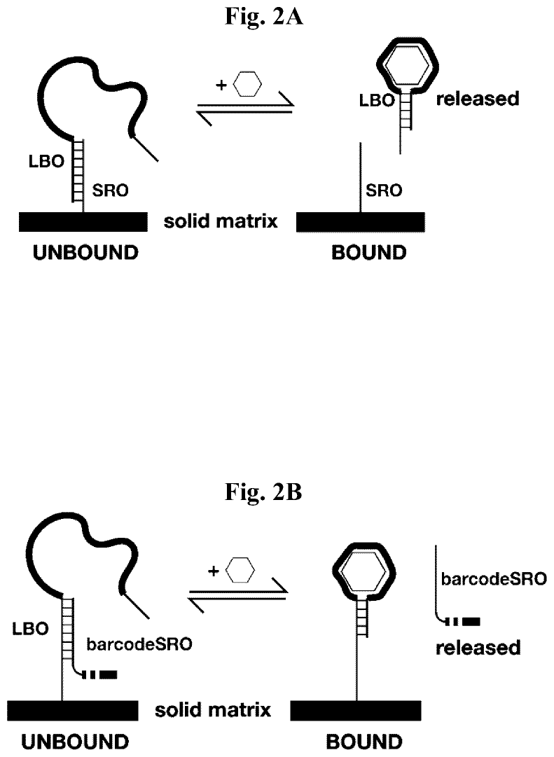

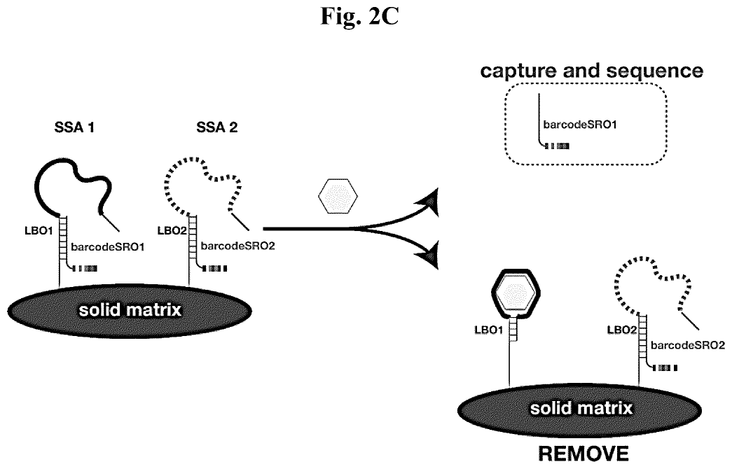

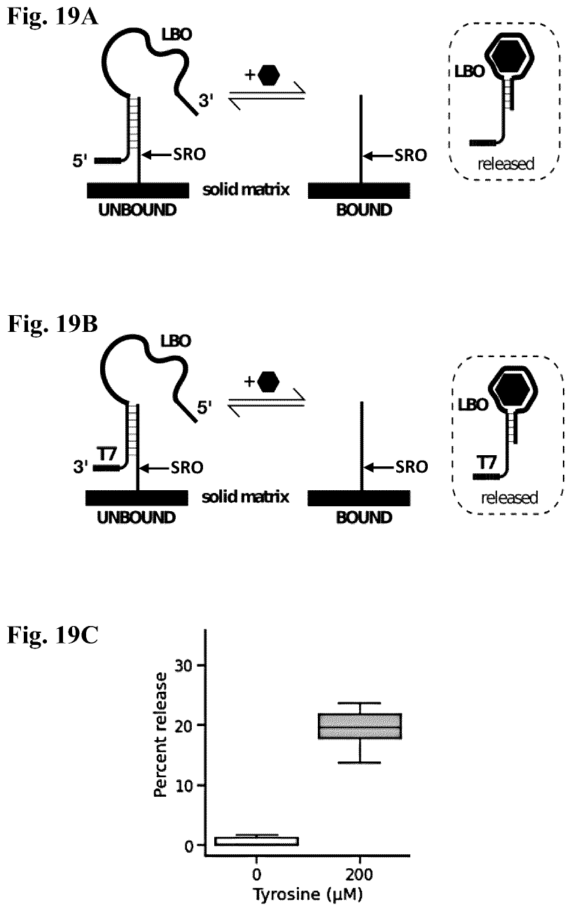

In a first aspect, described herein is a system for detecting one or more target ligands in a fluid sample, the system comprising one or more ligand-sensing complexes. In some embodiments, each ligand-sensing complex is specific for a target ligand and may comprise a ligand-binding oligonucleotide (LBO) hybridized to a corresponding barcoded short-release oligonucleotide (SRO) to form an LBO/SRO pairing. In some embodiments, the LBO comprises a ligand-binding region that specifically binds to the target ligand operably linked to an SRO hybridization region that is sufficiently complementary to a corresponding LBO hybridization region comprised in the SRO to enable formation of the LBO/SRO pairing (e.g., by conventional base pairing). In some embodiments, the bimolecular LBO/SRO structure may include a structure-switching aptamer (SSA) and can exist in two distinct states (e.g., as shown in FIGS. 2B and 19A). In the first state, in the absence of ligand, the LBO and SRO are paired (e.g., conventional base pairing) in a stable bimolecular hybrid. In the second state, ligand binding causes a conformational change which drives LBO-LBO self-interaction and dissociation of the LBO/SRO pairing.

In some embodiments, the base composition of the hybridization region can be altered to affect the melting temperature and the strength of the LBO/SRO pairing. In some embodiments, the SRO or LBO further comprises a detectable marker, preferably an amplifiable marker such as a nucleic acid barcode, ribozyme, and/or primer sequence or region, which is informative with respect to the target ligand recognized by the ligand-sensing complex. In some embodiments, each marker may be unique with respect to a recognized ligand and/or ligand-sensing complex species. Binding of the target ligand to the ligand-binding region of the LBO drives a conformational change triggering the dissociation of the SRO/LBO pairing, thereby enabling the released barcode region to be used as a readout for the presence or concentration of the target ligand in the fluid sample. In some embodiments, the LBO may be immobilized to a solid matrix and the barcode region may be comprised in the SRO, which is released into solution upon ligand binding to the ligand-sensing complex (FIG. 2B). In some embodiments, the SRO may be immobilized to a solid matrix and the barcode region may be comprised in the LBO, which is then released into solution upon ligand binding to the ligand-sensing complex (FIG. 19A).

In some embodiments, other detectable markers may be employed in the system described herein in the place of, or in addition to the barcode regions described herein. Examples of such markers include fluorescent (e.g., including fluorescence/quencher systems or Fluorescence Resonance Energy Transfer (FRET) systems), luminescent, colorimetric, or other signal-based markers. The use of such signal-based markers has been conventionally favored in the field of aptamer-based detection systems because of their ability to provide more rapid feedback as to ligand binding without the need for additional processing steps. For multiplex systems designed to detect a plurality of target ligands in a single minimal sample volume, the utilization of DNA barcode regions as markers unique for each ligand-sensing complex is preferred despite the additional processing steps and time required for their capture, amplification, and/or detection.

In contrast to many conventional ligand detection systems employed with SSAs that result in the same readout for all released SROs, the barcode embodiments described herein enable individually measuring a plurality of SROs in the same volume since the SROs are distinguishable by their individual barcodes. Each SSA has a unique ligand binding specificity (e.g., one SSA may see target 1, another may see target 2)—however if every SSA has the same functional readout such as the same fluorescent reporter or a change in conductance, it is impossible to distinguish which SSA has detected its target if they are in the same volume. This means that it is impossible, using conventional detection systems, to multiplex many tens, hundreds, thousands, or millions of SSAs in the same physical volume in the same assay/reaction since their readouts cannot be distinguished. The barcode SRO embodiments described herein address multiplexing challenges—each SSA has a different SRO with a different barcode. This enables one to read out the binding of many tens, hundreds, thousands, or millions of SSAs in the same volume by detecting which barcode SROs were released. The released barcode SROs can be identified uniquely using any technique that reads out DNA sequence including but not limited to any type of DNA sequencing (such as Illumina™ or nanopore sequencing) or hybridization-based methods such as DNA microarrays.

As used herein, the expression “ligand” or “target ligand” refers to any molecule that may be specifically bound by an aptamer (e.g., a nucleic acid aptamer). In some embodiments, the target ligands described herein may comprise a small molecule, protein, peptide, amino acid, antigen, fatty acid, monosaccharide, disaccharide, oligosaccharide, polysaccharide, metabolite, cytokine, chemokine, drug or drug metabolite, or any combination thereof.

As used herein, the expression “fluid sample” refers to any sample having or suspected of having a target ligand described herein that is suitable for detection via the system described herein. In some embodiments, the fluid sample may be a complex mixture of analytes. In some embodiments, the fluid sample may be (but is not limited to) a biological sample, such as from tears, blood, plasma, urine, spinal fluid, cell culture medium, cell lysate, or cellular cytoplasm. In some embodiments, the fluid sample may be an environmental sample, sewage sample, or industrial sample. In some embodiments, this could also be a food sample, or any other consumer goods, including but is not limited to the dairy industry, wine, etc.

In some embodiments, the ligand-binding region of the LBO described herein may comprise or be derived from an aptamer, such as a structure-switching aptamer. As used herein, “structure-switching aptamers” refer to oligonucleotide or peptide molecules that specifically bind to a target molecule, and upon binding of the target molecule, exhibit a conformational change. In some embodiments, the LBO may be of any sequence length or size enabling it to bind to its target ligand. In some embodiments, the LBO may be at least a 10, 20, 30, 40, 50, 60, 70, 80, 90, 100, 110, 120, 130, 140, 150, 160, 170, 180, 190, or 200-mer. In some embodiments, the LBO may be modified to increase the size of the ligand binding region. For example, the LBO may comprise one or more spacer sequences enabling the ligand-binding region to properly interact with and recognize its target ligand. In some embodiments, the ligand-binding region of the LBO described herein may comprise or be derived from an aptamer made up of polymeric organic molecules (including but not limited to peptides, peptoids, lipids, polysaccharides, or any combination thereof either that exists naturally or is artificially produced) fused to an oligonucleotide comprising an SRO hybridization region, to the extent that ligand binding to the peptide aptamer triggers a release of the SRO.

In some embodiments, the LBO and/or SRO described herein may be composed (in part or in whole) of DNA, RNA, or modified or synthetic nucleotides (such as XNA), or any combination thereof. In the context of LBO ligand-binding regions comprising or derived from peptide aptamers, the peptide aptamers may be partially or completely composed of a sequence comprising any naturally-occurring, modified, or synthetic amino acid.

In some embodiments, the LBO or SRO described herein may comprise an affinity tag (e.g., to facilitate immobilization on a solid matrix, removal or sequestration, or to capture and barcode detection).

In some embodiments, any affinity tag suitable for capturing/sequestering oligonucleotides or oligonucleotide complexes may be employed. Examples of affinity tags include biotin tags, epitope tags, polyhistidine tags and the like, and these may be coupled to the SRO directly. Alternatively, the barcode region comprised in the SRO or LBO may be purified or sequestered using hybridization to a second DNA molecule using conventional base pairing. Since each SRO or LBO has a unique barcode, this allows individual affinity purification/sequestration of any single or collection of the released barcode regions with molecules that specifically base-pair to any single or collection of barcodes. In this way, it is possible to purify/sequester all released barcode regions via a common affinity reagent or any single or any subset or released barcode regions using specific affinity reagents that base pair with their specific barcodes.

In some embodiments, the barcode region of the SRO described herein may be designed, manufactured, or adapted to facilitate capture and/or sequencing. For example, a portion of the barcode region may comprise a nucleotide sequence that facilitates priming (e.g., is hybridized by an amplification or sequencing primer, or a primer that can be used to direct transcription of the barcode, transcription of a detectable marker (e.g., Baby Spinach), or to be used as a primer for primer-based detection methods such as PCR or rolling circle amplification). In some embodiments, the priming portion may comprise one or more sequences shared in barcode regions from more than one different type of ligand-sensing complex, thereby enabling amplification of multiple released SROs via the same amplification primer.

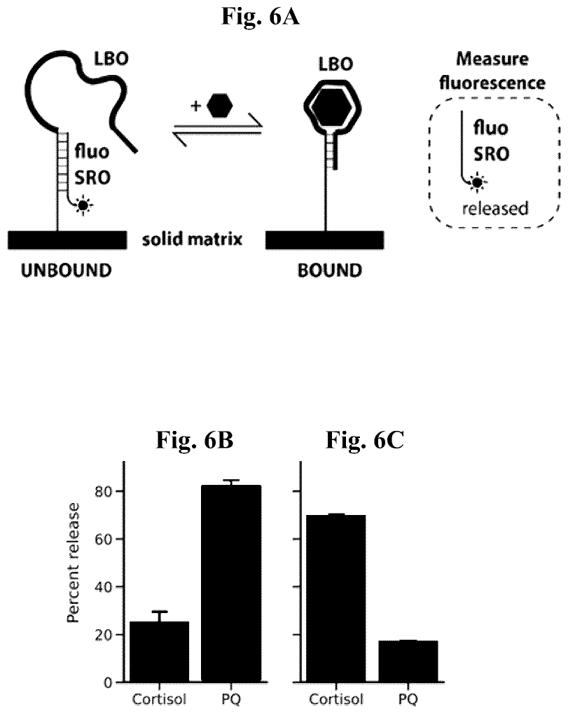

In some embodiments, the ligand-sensing complex described herein may be immobilized (e.g., on a solid matrix or solid support) via binding of the LBO to the matrix such that the LBO remains immobilized upon release of the barcoded SRO following target ligand binding (e.g., as shown in FIG. 6A). In some embodiments, the ligand-sensing complex described herein may be immobilized (e.g., on a solid matrix or solid support) via binding of the SRO to the matrix such that the SRO remains immobilized upon release of the barcoded LBO following target ligand binding (e.g., as shown in FIG. 19A). In some embodiments, any suitable immobilization strategy may be employed which facilitates physical separation/removal of the barcode region released from the ligand-sensing complex upon ligand binding in the context of the system described herein.

In some embodiments, the system described herein may comprise a mixture of a plurality of the same or different ligand-sensing complexes that are immobilized to the same solid matrix or solid support, but that are not physically or spatially separated or arranged from each other (e.g., at predetermined or readily discernable positions, such as on an array), thereby reducing the volume of fluid sample required.

In some embodiments, ligand-sensing complexes described herein may be immobilized or bound onto a solid matrix, such as but not limited to a bead or column or the well of plate, or on a lateral flow strip. In some embodiments, the ligand-sensing complexes may be immobilized to the matrix by binding of the LBO or SRO to the matrix via a matrix-binding portion. In some embodiments, the matrix-binding portion of the LBO or SRO is modified for binding to the matrix. Modifications may include but are not limited to biotinylating the LBO or SRO and binding to a streptavidin-coated matrix.

In some embodiments, the LBO-SRO bimolecular hybrid can be distinguished from an LBO that has released an SRO (or vice versa) following ligand binding using enzymes that recognize and specifically cleave the SRO-LBO hybridized region.

In some embodiments, the system described herein may comprise a plurality of the same or different ligand-sensing complexes each having similar LBO/SRO hybridization characteristics, thereby providing different SROs with the same or similar release profiles. In some embodiments, similar LBO/SRO hybridization characteristics may be achieved by having LBO/SRO hybridization regions with similar melting temperatures (Tₘ) that do not differ by more than 10, 9, 8, 7, 6, 5, 4, 3, 2, or degrees). In some embodiments, the LBO and SRO hybridization regions of each of the ligand-sensing complexes in the system may have the same nucleotide composition and/or the same nucleotide sequence.

In a further aspect, described herein is a method for preparing one or more ligand-sensing complexes having correctly paired LBO/SRO molecules. The method generally comprises providing one or more unimolecular polynucleotides, each unimolecular polynucleotide comprising the nucleotide sequences of both the LBO and the SRO separated by a cleavage site (e.g., at an abasic site, a photocleavable site, or an enzymatic cleavage site) positioned therebetween. The complementary LBO and SRO hybridization regions are allowed to hybridize to each other before the unimolecular polynucleotide is cleaved at the cleavage site, thereby producing the one or more ligand-sensing complexes having separate LBO and SRO molecules that are correctly paired. In some embodiments, the one or more unimolecular polynucleotides may be immobilized on a solid matrix prior to or following the cleavage such that ligand-sensing complexes remain immobilized following cleavage. In some embodiments, a mixture of different unimolecular polynucleotides (i.e., designed to recognize different target ligands) are cleaved together in the same reaction solution, thereby producing a plurality of different ligand-sensing complexes in parallel, each having correctly paired LBO/SRO molecules. In some embodiments, the LBO and the SRO of each ligand-sensing complex may comprise a terminal end resulting from cleavage (e.g., at an abasic site, a photocleavable site, or an enzymatic cleavage site) of a unimolecular polynucleotide comprising the nucleotide sequences of both the LBO and the SRO.

In a further aspect, described herein is a unimolecular polynucleotide as described herein for use in preparing a ligand-sensing complex as described herein. In a further aspect, described herein is the use of a unimolecular polynucleotide as described herein for preparing a ligand-sensing complex as described herein.

In some embodiments, the system described herein is a multiplexed system comprising a plurality of different ligand-sensing complexes, each complex being designed to release a different barcode upon binding of a different target ligand, thereby enabling detection of a plurality of different target ligands from a single fluid sample volume.

As used herein, the term “multiplexed” refers to a system that allows for the simultaneous detection of a plurality of distinct target ligands in a single assay volume (e.g., at least 2, at least 6, at least 10, at least 20, at least 30 target molecules). In multiplexed systems described herein, it is preferred that there is minimal to no crosstalk between different ligand-sensing complexes such that target ligand binding to one ligand-sensing complex does not displace the SRO from a separate unrelated ligand-sensing complex. In some embodiments, the multiplexed systems may comprise detection of distinct ligands or detection of the same target ligands at different concentrations.

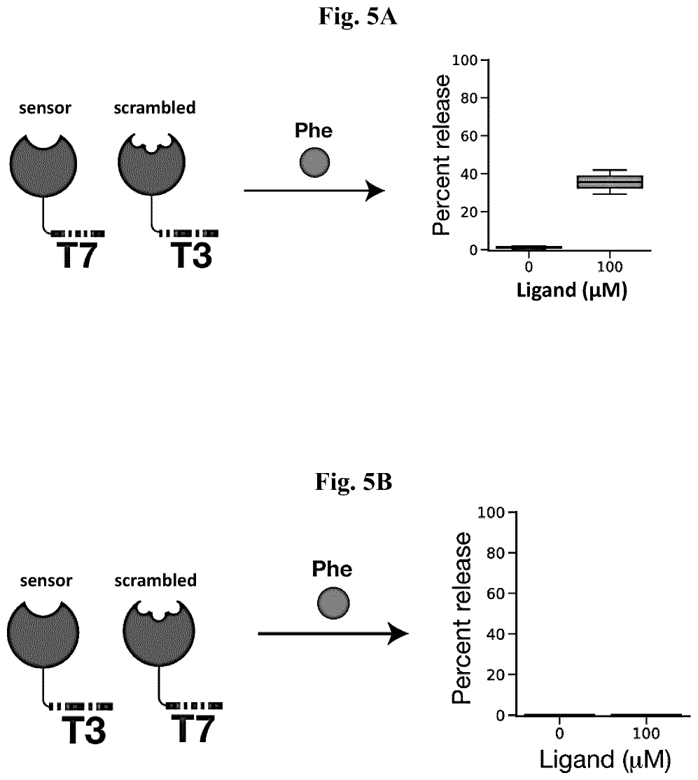

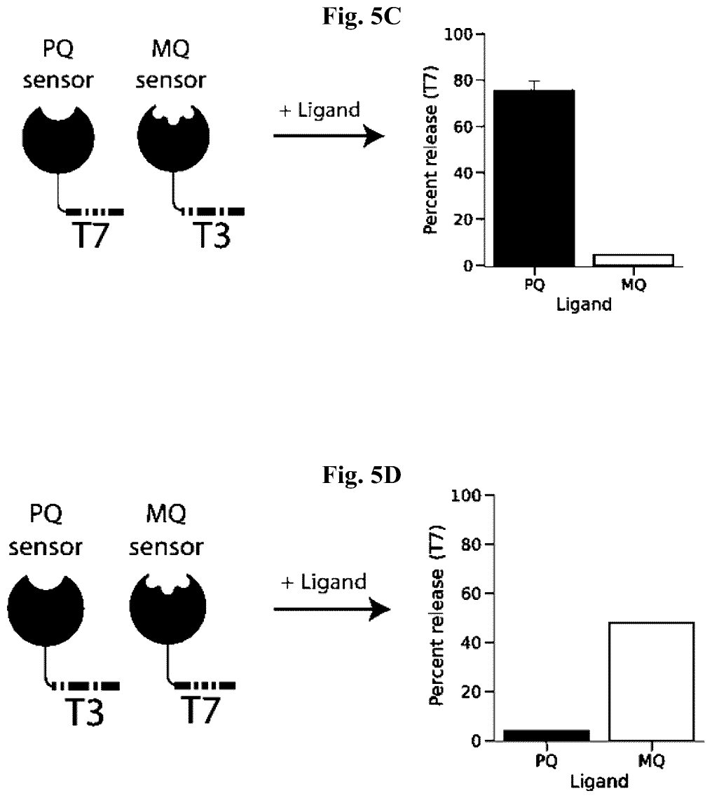

In some embodiments, the system described herein is a multiplexed system comprising a plurality of different barcoded ligand-sensing complexes heterogeneously mixed and/or hybridized to the same surface (e.g., to the same bead or solid matrix) in a location agnostic fashion (e.g., without spatial arrangement such as an array or multiwell plate) such that the particular locations of the complexes on the surface are uncontrolled or not readily determinable. Such architectures are made feasible when there is negligible crosstalk between different barcoded ligand-sensing complexes (e.g., as shown in Example 5 and FIGS. 5A to 5D) and are not feasible when employing only signal-based markers. Furthermore, the results in Example 5 and FIG. 6A to 6C suggest that the use of different barcoded ligand-sensing complexes exhibit potentially lower non-specific signal than when different fluorescently labeled ligand-sensing complexes are employed.

While the use of signal-based markers (e.g., fluorescence, luminescent, colorimetric, and/or electric signal-based markers) have been conventionally favored in aptamer-based detection systems because they may provide more rapid feedback as to ligand binding (e.g., potentially in real-time), the use of such markers limits the ability to multiplex. The number of ligands detectable in parallel using only signal-based markers is greatly hindered by the limited number of discrete signal-based markers available (e.g., fluorophores with distinct, non-overlapping emission spectra). A conventional solution to such a drawback has been to use the same signal-based marker for different aptamer sensors but to physically or spatially separate or arrange the different aptamer sensors with respect to a solid matrix (e.g., on an array), in individual droplets, or in different wells (e.g., US 2019/0242030 A1). In such a setup, the identity of the ligand that is bound to the aptamer is conferred by the position of signal emitted with respect to the solid matrix and not by the nature of the signal itself (e.g., emission wavelength of fluorophore). However, a major drawback of spatially separating the aptamer sensors is that greater sample volumes are required, making such architectures not suitable for multiplex assays in the context of minimal sample volumes.

In some embodiments, the system described herein may comprise multiple species of ligand-sensing complexes that bind to the same target ligand, wherein each species comprises LBOs having identical ligand-binding regions but that differ in their non-ligand binding structural elements, thereby providing a plurality of LBO species that bind to the target ligand at different but overlapping dynamic ranges such that the overall dynamic range of the system is greater than that of a single LBO species. In some embodiments, the LBO species comprise aptamers having stem-loop structures, and each of the LBO species differ in their stem structures (Example 15).

In some embodiments, the system described herein may further comprise an inert macromolecular crowding agent for admixture with the fluid sample at a concentration sufficient to increase the ligand-sensing complexes' sensitivity and/or dynamic range with respect to its target ligand, as compared to in the absence of the inert macromolecular crowding agent. In some embodiments, the macromolecular crowding agent is or comprises one or more of polyethylene glycol; a neutral branched hydrophilic polysaccharide (e.g., Ficoll); dextran; a protein (e.g., albumin); or other inert macromolecule. In some embodiments, the concentration of the macromolecular crowding agent is at least about 0.5, 0.6, 0.7, 0.8, 0.9, 1, 1.5, 2, 2.5, 3, 3.5, 4, 4.5, or 5% to about 6, 6.5, 7, 7.5, 8, 8.5, 9, 9.5, 10, 11, 12, 13, 14, 15, 16, 18, 19, 20, 21, 22, 23, 24, 25, 26, 27, 28, 29, 30, 35, 40, 45, 50, 55, 60, 65, or 70% (w/v).In a further aspect, described herein is a method for detecting or measuring a ligand in a fluid sample, the method comprising: providing a system as described herein; contacting the system with the fluid sample; and detecting the released barcoded SRO as a readout for the presence or concentration of the target ligand in the fluid sample. In some embodiments, the detecting step may comprise capturing the released barcoded SRO; amplifying the barcode region of the released SRO; sequencing the barcode region of the released SRO; or any combination thereof.

In another aspect, the present description relates to a kit for the detection of one or more target ligands, the kit comprising one or more ligand-sensing complexes, LBO molecules, SRO molecules, and/or unimolecular polynucleotides describe herein. The kit may further comprise one or more reagents or buffers, and/or instructions for use.

In a further aspect, described herein is an aptamer-based detection system having improved (e.g., expanded or broadened) dynamic range for a target ligand, the system comprising a plurality of ligand-binding oligonucleotide (LBO) species that bind to the same target ligand. In some embodiments, the plurality of LBO species comprises LBO species having different ligand-binding regions that specifically bind to the target ligand but with different affinities. In some embodiments, the plurality of LBO species comprises LBO species having identical ligand-binding regions that specifically bind to the target ligand but that differ in their non-ligand binding structural elements. In some embodiments, the plurality of LBO species comprises both: (a) LBO species having different ligand-binding regions that specifically bind to the target ligand but with different affinities, and (b) LBO species having identical ligand-binding regions that specifically bind to the target ligand but that differ in their non-ligand binding structural elements (Example 15). The resulting aptamer-based detection system thus comprises a plurality LBO species that bind to the same target ligand at different but overlapping dynamic ranges such that the overall dynamic range of the system is greater than that of a single LBO species. In some embodiments, the aptamer-based detection system may employ or comprise structure-switching aptamers. In some embodiments, the LBO species (e.g., sharing identical of very similar ligand-binding regions) may comprise aptamers having stem-loop structures, and wherein each of the LBO species differ in their stem structures (i.e., non-ligand binding structural elements).

In a further aspect, described herein is the use of an inert macromolecular crowding agent to increase the sensitivity and/or dynamic range of an aptamer for its ligand, as compared to in the absence of the inert macromolecular crowding agent. As used herein, the term “inert” in the expression “inert macromolecular crowding agent” means that the macromolecular crowding agent does not interfere with the detection of the target ligands of interest by the aptamer. In some embodiments, described herein is a method for increasing the sensitivity and/or dynamic range of an aptamer for its ligand, the method comprising: providing a sample comprising or suspected of comprising a ligand of interest; contacting the sample with an aptamer that binds to the ligand of interest in the presence of a concentration of an inert macromolecular crowding agent sufficient to increase the aptamer's sensitivity and/or dynamic range with respect to its ligand, as compared to the aptamer's sensitivity and/or dynamic range in a corresponding sample lacking the inert macromolecular crowding agent. In some embodiments, the macromolecular crowding agent is or comprises one or more of: polyethylene glycol; a neutral branched hydrophilic polysaccharide (e.g., Ficoll); dextran; a protein (e.g., albumin); or other inert macromolecule. In some embodiments, the concentration of the macromolecular crowding agent is at least about 0.5, 0.6, 0.7, 0.8, 0.9, 1, 1.5, 2, 2.5, 3, 3.5, 4, 4.5, or 5% to about 6, 6.5, 7, 7.5, 8, 8.5, 9, 9.5, 10, 11, 12, 13, 14, 15, 16, 18, 19, 20, 21, 22, 23, 24, 25, 26, 27, 28, 29, 30, 35, 40, 45, 50, 55, 60, 65, or 70% (w/v). In some embodiments, the aptamer is a structure-switching aptamer. In some embodiments, the aptamer is a ligand-sensing complex as described herein, or is comprised in a system as described herein.

### EXAMPLES

### Example 1: Materials and Methods

### Reagents

All oligonucleotides were synthesized by Integrated DNA Technologies (IDT) (Coralville, IA) and dissolved in nuclease-free water at a concentration of 100 μM. The oligonucleotide sequences used were adapted from Yang et al., 2014; Coonahan et al., 2021; Song et al., 2012; and Warner et al., 2014. Oligonucleotide sequences may be biotinylated or modified to include spacers, such as IntSpacer18™ (“iSp18”; an 18-atom hexa-ethyleneglycol spacer; IDT) or idSp (Int 1′,2′-Dideoxyribose (dSpacer™); IDT). Oligonucleotide sequences used in the studies described herein include:

TABLE 2
Oligonucleotide sequences used in Examples 1-9

| | |
|---|---|
| T7 Baby Spinach | 5′ GGTGCTCACACTCTACTCAACAGTAGCGAACTACTGGACCCGTCCTTCACCTATAGTGAGTCGT |
| transcription template | ATTAGACTC 3′ |
| (SEQ ID NO: 1): | |
| Phenylalanine LBO | 5′ Biotin-iSp18- |
| (SEQ ID NO: 2): | CTCTCGGGACGACGGACGCTAATCTTACAAGGGCGTAGTGTATGTCGTCCC 3′ |
| Scrambled Phe LBO | 5′ Biotin-iSp18- |
| (SEQ ID NO: 3): | CTCTCGGGACGACAGGAGACTTAGATGTTATATCAGGCGTCGCGTCGTCCC 3′ |
| Tyrosine LBO | 5′ Biotin-iSp18-CTCTCGGGACGACGGCCCGATCTCAGAGTAGTCGTCCC 3′ |
| (SEQ ID NO: 4): | |
| T7 SRO | 5′ GTCGTCCCGAGAGTAATACGACTCACTATAGG 3′ |
| (SEQ ID NO: 5): | |
| T3 SRO | 5′ GTCGTCCCGAGAGAATTAACCCTCACTAAAGG 3′ |
| (SEQ ID NO: 6): | |
| Piperaquine LBO | 5′ Biotin- |
| (SEQ ID NO: 7): | TAAGATGACTCGGCAACGGCACACGCTAGTTGCGCCGAATGGCCGTGAAACCGTTGCC 3′ |
| Piperaquine T7 SRO | 5′ TGCCGTTGCCGAGTCTAATACGACTCACTATAGG 3′ |
| (SEQ ID NO: 8): | |
| Piperaquine T3 SRO | 5′ TGCCGTTGCCGAGTCAATTAACCCTCACTAAAGG 3′ |
| (SEQ ID NO: 9): | |
| Mefloquine LBO | 5′ Biotin- |
| (SEQ ID NO: 10): | TAAGATCTCTCGGGACGACGGCAGTCTATACCCCGTATCGCCGAAGGTTGTCGTCCC 3′ |
| Mefloquine T7 SRO | 5′ CCGTCGTCCCGAGAGTAATACGACTCACTATAGG 3′ |
| (SEQ ID NO: 11): | |
| Mefloquine T3 SRO | 5′ CCGTCGTCCCGAGAGAATTAACCCTCACTAAAGG 3′ |
| (SEQ ID NO: 12): | |
| Ampicillin LBO | 5′ Biotin-iSp18-CTCTCGGGACGACGCGGGCGGTTGTATAGCGGGTCGTCCC 3′ |
| (SEQ ID NO: 13): | |
| Ampicillin Scrl LBO | 5′ Biotin-TAAGATGACTCGCGAGAGCGCGGGCGGTTGTATAGCGGGCTCTCGC 3′ |
| (SEQ ID NO: 14): | |
| Ampicillin Scr2 LBO | 5′ Biotin-TAAGATGACTCGGCAACGGGCGGGCGGTTGTATAGCGGCCGTTGCC 3′ |
| (SEQ ID NO: 15): | |
| Ampicillin Scr1 T7 SRO | 5′ GCTCTCGCGAGTCTAATACGACTCACTATAGG 3′ |
| (SEQ ID NO: 16): | |
| Ampicillin Scr2 T7 SRO | 5′ CCGTTGCCGAGTCTAATACGACTCACTATAGG 3′ |
| (SEQ ID NO: 17): | |
| Tryptophan SSA | 5′ Biotin-iSp18- |
| (SEQ ID NO: 18): | CTCTCGGGACGACCGCGGTAGTCTTAACCTAAAGCGGTGTCAGGTCGTCCC-idSp- |
| | GTCGTCCCGAGAGAATTAACCCTCACTAAAGG 3′ |
| Scrambled Trp SSA | 5′ Biotin-iSp18- |
| (SEQ ID NO: 19): | CTCTCGGGACGACTATGCCGGAGTAGCGGATGCCACCAGTTATGTCGTCCC-idSp- |
| | GTCGTCCCGAGAGAATTAACCCTCACTAAAGG 3′ |
| Tyr 9A LBO | 5′ Biotin-iSp18-CTCTCGGGACGACGGCCCGAAAAAAAAAAGTAGTCGTCCC 3′ |
| (SEQ ID NO: 20): | |
| Tyr 9T LBO | 5′ Biotin-iSp18-CTCTCGGGACGACGGCCCGTTTTTTTTTAGTAGTCGTCCC 3′ |
| (SEQ ID NO: 21): | |

Stock solutions of phenylalanine (25 mM), tryptophan (25 mM), ampicillin (100 mM) and pentamethylcyclopentadienyl rhodium dichloride dimer [Cp*RhCl₂]₂ (5 mM) were made in nuclease-free water while the stock solution of tyrosine (2 mM) was made directly in binding buffer (20 mM HEPES (pH 7.5), 1 M NaCl, 10 mM MgCl₂, 5 mM KCl). Stock solutions of piperaquine (PQ) (1 mg/mL) and mefloquine (500 μg/mL) were made in 5% methanol. The stock solution of DFHBI-1T (20 mM) was made in DMSO.

### Ligand Binding Assay

For experiments with phenylalanine, tyrosine and tryptophan, the amino acids were complexed with Cp*Rh(III) at a final concentration of 100 μM Cp*Rh(III) and varying concentrations of amino acids, and incubated together at room temperature for >45 min. All dilutions were made in binding buffer as per Yang et al. (2014).

For each sample, 5 μL of Dynabeads MyOne™ Streptavidin C1 magnetic beads (Invitrogen) were washed 3× in Bind & Wash buffer as per manufacturer's protocol, and finally resuspended in 10 μL binding buffer. 25 pmol of LBO and 125 pmol of SRO (10 μL total, diluted in binding buffer) were heated at 95° C. for 5 min and slowly cooled to 25° C. The oligos were then added to the resuspended beads and incubated on a rotator for 30 min at room temperature. The beads were then washed 2× with 100 μL binding buffer and resuspended in 20 μL of the same buffer and incubated for another 45 min. The beads were then washed 1× and resuspended with 20 μL of the amino acid-Cp*Rh(III) complex. Cp*Rh(III) alone was used as the negative control. The samples were incubated for 45 min on a rotator at room temperature and the supernatant was collected. The beads were then resuspended in 20 μL of strand separation buffer (20 mM HEPES (pH 7.5), 300 mM NaCl), heated at 95° C. for 4 min, and the supernatant sample collected (‘remainder’ sample). Percent release is the calculated as:

$$\frac{supernatant RFU}{supernatant RFU + remainder RFU} .$$

For experiments with piperaquine and mefloquine, the beads and oligonucleotides were prepared as described above, except that PBS with 4 mM MgCl₂ was used as the binding buffer, and PBS was used as the strand separation buffer.

For the LB+PQ experiments, the LB broth (BioShop) was diluted 1:2 in PBS and spiked with various amounts of piperaquine. SSAs were then incubated in these mixtures. LB medium with no piperaquine added was used as the negative control. For lysate+PQ experiments, lysates of worms were diluted 1:5 in PBS and spiked with various amounts of piperaquine. Lysate with no piperaquine added was used as the negative control.

For LB+Amp experiments, the LB broth was diluted 1:5 in binding buffer and spiked with various amounts of ampicillin. To maintain similar salt conditions as used for previous SSA experiments, NaCl, MgCl₂ and KCl were added to the LB medium at a final concentration of 1 M, 10 mM and 5 mM respectively. LB medium with no ampicillin added was used as the negative control.

For samples with a T7 SRO, 1.5 μL of the 20 μL supernatant sample was added to 5 μL of nuclease-free water and 2 μL of 10 μM Baby Spinach transcription template. Oligos were mixed and heated at 95° C. for 5 min and slowly cooled to 25° C. Reaction buffer, NTPs (2 mM each) and T7 RNA polymerase were then added to the sample for a total volume of 20 μL and incubated at 37° C. for 2 h. After transcription, the sample was then heated at 90° C. for 2 min before incubation on ice for >3 min. 30 μL of water and 5 μL of 100 μM DFHBI-1T (diluted in Tris-HCl buffer: 40 mM Tris-HCl (pH 8.0), 5 mM MgCl2, 125 mM KCl) were added to 15 μL of the sample, and the sample was then heated to 65° C. for 5 min and slowly cooled to room temperature (as per Okuda et al., 2017). Each sample was then transferred to a 96-well plate and the fluorescence intensity for each sample was measured using a FLUOstar™ Omega microplate reader (excitation 485 nm, emission 520 nm).

For samples with a 6-FAM or TEX615 SRO, 20 μL of the sample was added to 35 μL of water and fluorescence was directed measured using the microplate reader (6-FAM: ex 485 nm, em 520 nm; TEX615: ex 584 nm, em 620 nm).

### Cleavage of Abasic Site

A dSpacer (IDT) was used as the abasic site. For each sample, 25 pmol of SSAs were folded in 1× NEBuffer™ 3 (NEB) by heating at 95° C. for 5 min followed by slow cooling to 25° C. Endonuclease IV (NEB) was then added to the sample and the reaction mix was incubated at 37° C. overnight. After incubation, the sample was then added to the dynabeads (described above), along with additional NaCl and KCl such that their final concentrations were at 1 M and 5 mM respectively. The binding assay was then carried out the same way as above.

### T7 Baby Spinach Transcription Assay

1.5 μL of the 20 μL supernatant sample was added to 5 μL of nuclease-free water and 2 μL of 10 μM Baby Spinach transcription template. Oligos were mixed and heated at 95° C. for 5 min and slowly cooled to 25° C. Reaction buffer, NTPs (2 mM each) and T7 RNA polymerase were then added to the sample for a total volume of 20 μl and incubated at 37° C. for 2 h.

After transcription, the sample was then heated at 90° C. for 2 min before incubation on ice for >3 min. 30 μL of water and 5 μl of 100 μM DFHBI-1T (diluted in Tris-HCl buffer: 40 mM Tris-HCl (pH 8.0), 5 mM MgCl₂, 125 mM KCl) were added to 15 μL of the sample, and the sample was then heated to 65° C. for 5 min and slowly cooled to room temperature (as per Okuda et al., 2017). Each sample was then transferred to a 96-well plate and the fluorescence intensity for each sample was measured using a FLUOstar™ Omega microplate reader (excitation 485 nm, emission 520 nm).

### Drug Treatment and Worm Lysis

C. elegans worms (wild-type N2 and bus-5(br19) strains) were grown and maintained on NGM agar plates seeded with OP50 bacteria. Strains were provided by the CGC, which is funded by NIH Office of Research Infrastructure Programs (P40 OD010440).

For drug treatments, OP50 cultures were heat-killed at 65° C. for 30 min, spun down, then concentrated 2-fold in NGM media. 1600 μL of the suspended cultures was then added to 200 μL of mixed stage bus-5(br19) worms in M9, along with 200 μL of 10× concentrated drug. The worms were then incubated with the drugs for 7 h in a 20° C. shaker, with piperaquine and mefloquine added at a final concentration of 100 μg/ml and 50 μg/ml respectively. Worms were treated with 5% methanol in lieu of drugs as the negative control. After 7 h drug treatment, the worms were washed 3× in M9 followed by 1× in PBS. The worm pellet was then flash frozen and stored at −80° C.

To lyse the worms, the frozen pellets were ground with a pestle until the pellet defrosted. Extraction solvent (8:1:1 ratio of methanol:chloroform:water) was then added to the tubes at 3× the volume of the pellet. Samples were then vortexed and subjected to 3 freeze-thaw cycles. The tubes were then centrifuged at 13,200 rpm for 15 min and the supernatants were collected and stored at −80° C. until needed. The worm lysate used for the piperaquine spike-in experiment was collected from N2 worms that were not treated with any drugs.

### Transcription and Amplification of Barcode SRO (SEQ ID NO: 23)

The anti-sense sequence of a T7 promoter and a 12 nt random barcode sequence (1:1:1:1 A:C:G:T base ratio) is added downstream of the SSA stem hybridisation region. A constant 7 nt sequence known to increase transcription efficiency (Conrad et al. 2020) was also included just downstream of the T7 promoter and upstream of the barcode. An excess of T7 forward primer is then annealed to the barcode SRO and in vitro transcription was carried out for 6 h. The resulting products were then visualised on a 4% agarose gel.

For the Phe SSA experiment, the ligand binding assay was carried out as previously described. 1.5 μl of the 20 μl supernatant sample was then added to 2 μL of 10 μM T7 forward primer, followed by 6 h in vitro transcription.

### Two-Color Multiplexing Assay

The PQ LBO was paired with an SRO attached to a 6-FAM fluorophore (IDT) while the cortisol LBO was paired with an SRO attached to a TEX615 fluorophore (IDT). For the multiplexing experiment involving PQ and cortisol, two different stem variants were used for the PQ and cortisol SSAs, and equal amounts of PQ and cortisol SSAs bound to beads were mixed together. Ligand binding was then assayed in PBS supplemented with 10 mM Mg and 5% ethanol. SSAs were incubated with either PQ or cortisol for 45 min on a rotator at room temperature and the supernatant was collected. The beads were then resuspended in 20 μL of PBS, heated at 95° C. for 4 min, and the supernatant collected. After addition of 35 μl water to each sample, fluorescence was then measured using a FLUOstar™ Omega microplate reader at 2 different excitation/emission wavelengths for 6-FAM (ex 485 nm, em 520 nm) and TEX615 (ex 584 nm, em 620 nm).

Cortisol LBO (SEQ ID NO: 41):
5′ (Biotin)-
TAAGATCTCTCGGGACGACGCCCGCATGTTCCATGGATAGTCTTGACT
AGTCGTCCC 3′
6-FAM Piperaquine SRO (SEQ ID NO: 42):
5′ GCCGTTGCCGAGTCTA-(6-FAM) 3′
TEX615 Cortisol SRO (SEQ ID NO: 43):
5′ GTCGTCCCGAGAGT-(TEX615) 3′

Cleavage of Pre-SSAs with Nb.BstI

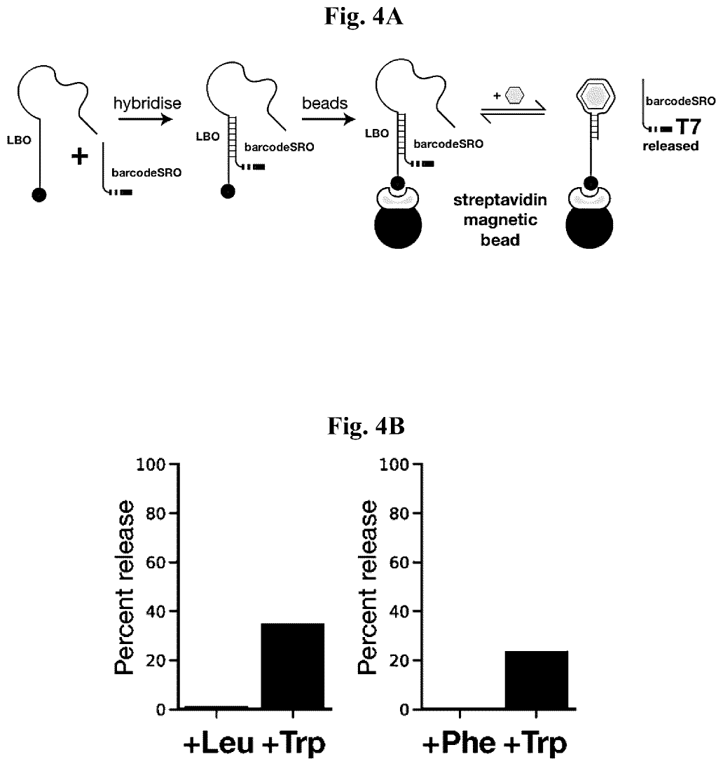

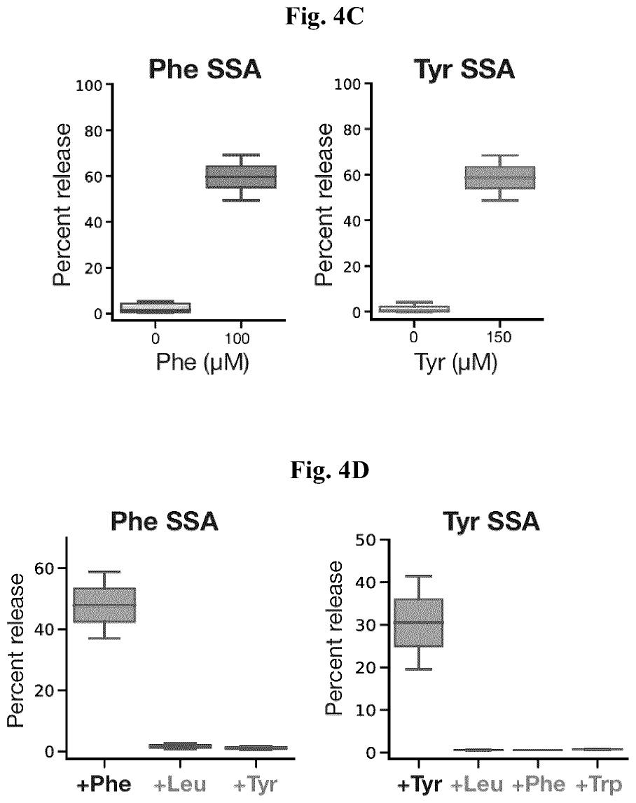

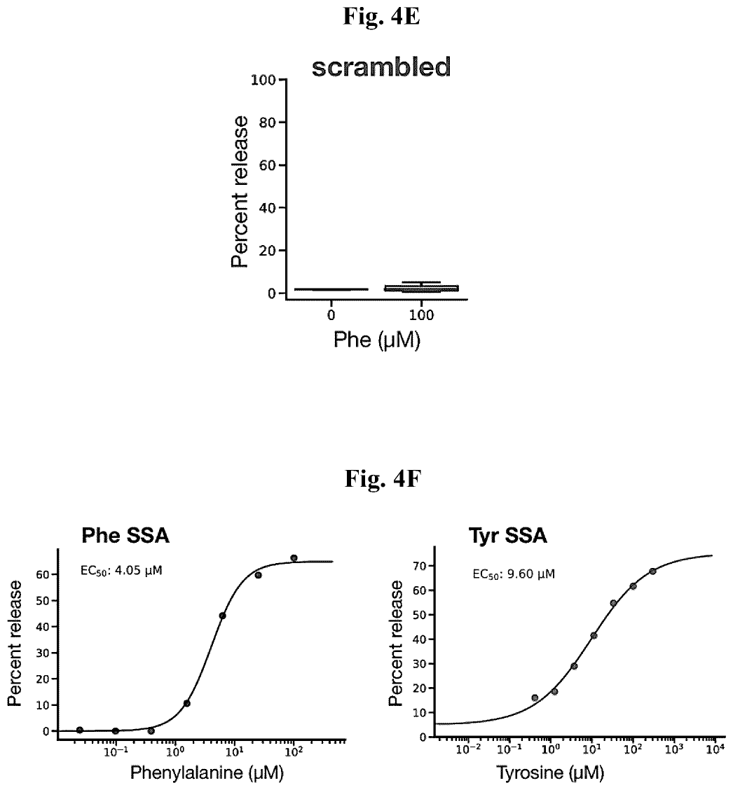

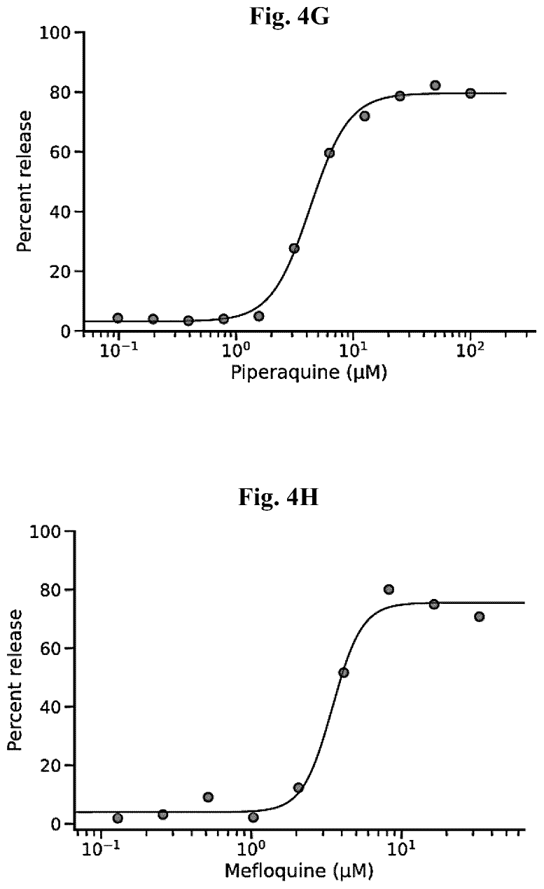

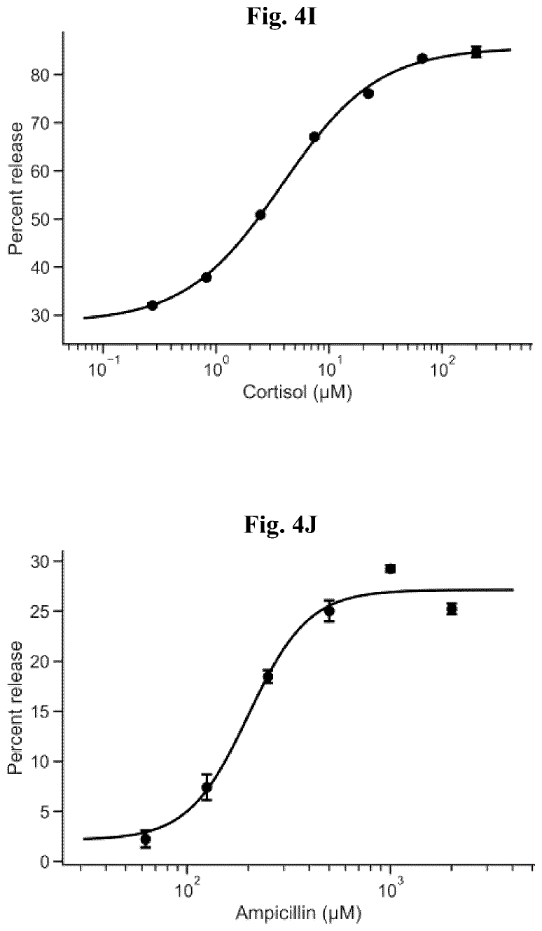

1.25 μL of 100 μM pre-SSA was added to 5 μL of CutSmart™ buffer (NEB) and 38.75 μL of water. The sample was then heated at 95° C. for 5 min and cooled to fold the pre-SSAs. 5 μL of Nb.BstI (NEB) was then added to the sample and the sample was incubated at 37° C. for 2 h. Samples were then boiled in loading buffer for 5 min before being run on a 15% TBE-Urea gel (BioRad) at 200 V for 45 min. The gel was then stained with SYBR™ Gold (Invitrogen) for 30 min before imaging. For FIG. 4, various cooling and enzyme incubation times were tested: (1) fast cooling on ice; 1 h cleavage, (2) slow cooling to room temperature (over 45 min); 1 h cleavage, (3) fast cooling on ice; 6 h incubation at 4° C.; 2 h cleavage, (4) slow cooling; 6 h incubation at 4° C.; 2 h cleavage, (5) slow cooling; 1.5 h cleavage. An input sample without any Nb.BstI added was used as a negative control.

Nb.BstI Piperaquine LBO (SEQ ID NO: 44):
5′ (Biotin)-
TAAGATGACTCGGGCAGTGCACACGCTAGTTGCGCCGAATGGCCGTGA
AACACTGCCC 3′
Nb.BstI Piperaquine SRO (SEQ ID NO: 45):
5′ TGCACTGCCCGAGTCTAATACGACTCACTATAGG 3′

### Selections Using N12 Controlled Complexity Libraries

For ampicillin selection experiments, we performed selections using an N12 sequence and an N4-T4-N4-T4-N4 sequence in the ligand-binding region. Both libraries were added in equimolar amounts and selections were performed as described elsewhere (Yang et al., 2014; Yang et al., 2016). After 1 round of selection with 100 μM ampicillin, the eluate was concentrated and amplified for sequencing. Library preparation was carried out using a Nextera DNA Flex kit (Illumina) followed by sequencing on the NextSeq500 with 20% PhiX spike-in. Enriched sequences were then clustered and sequences from each cluster were re-ordered as oligos in our standard barcode SSA configuration using a 6-FAM SRO readout. Dose response curves were the generated as described above.

Ampicillin A4 LBO (SEQ ID NO: 46):
5′ Biotin-
TAAGATGGCTCTCGGGACGACTATGTTTTGGGGTTTTTATAGTCGTCCC
GA 3′
Ampicillin E3 LBO (SEQ ID NO: 47):
5′ Biotin-
TAAGATGGCTCTCGGGACGACCAGTTTTTGGTTTTTTTGTGGTCGTCCC
GA 3′
Ampicillin G2 LBO (SEQ ID NO: 48):
5′ Biotin-
TAAGATGGCTCTCGGGACGACGTTTAATTTATTGTCGTCCCGA 3′
Ampicillin A4/E3/G2 6-FAM SRO (SEQ ID NO: 49):
5′ GTCGTCCCGAGAGCC/36-FAM/3′

### Varying Sequence Composition of Stems to Expand Dynamic Range

The various stem sequences tested are listed in Table 3. Sequences were scrambled but with the same % GC content and with lengths varied in some cases. Dose response curves were generated using the same binding assay and T7 SRO readout as described above. For each SSA variant, SRO release was normalised with 100% response corresponding to maximal SRO release and 0% response corresponding to the lowest SRO release for that variant. After curve-fitting, the dynamic range of each SSA is defined as between 20% to 80% of the normalised release response. Specific values extracted from the dose response curves are listed in Table 4.

### Addition of Macromolecular Crowding Reagents to Increase Sensor Sensitivity

For all experiments with macromolecular crowding reagents, a 6-FAM SRO was used to provide a direct fluorescence readout. Stock solutions of PEG 8000 (25%; BioShop) and Ficoll™-70 (20%; Sigma) were freshly made in binding buffer (20 mM HEPES (pH 7.5), 1 M NaCl, 10 mM MgCl₂, 5 mM KCl) for each experiment. PEG, Ficoll and drug titrations were made in the same binding buffer for all the binding assays. Ligand binding assays were then performed as described previously and fluorescence measured using a microplate reader (excitation 485 nm, emission 520 nm).

### Example 2: Methods to Generate Libraries of Barcoded Structure-Switching Aptamers

Structure switching aptamers (SSAs) are powerful tools for the detection of small molecules of interest. In broadest terms, when the correct small molecule ligand is bound by the ligand-binding region of the aptamer, this drives a conformational change which can be detected in a variety of ways. Different SSAs have different ligand binding specificities and thus this basic approach can be used to detect many different ligands. One method to detect the conformational change in an SSA due to ligand binding involves the dissociation of a bimolecular SSA into its two separate parts—the ligand binding oligo (LBO from here on) and a short release oligo (SRO from here on). The LBO and SRO interact via base-pairing, which is then disrupted by the conformational change following ligand binding. That dissociation can be detected using many possible methods, many of which commonly involve fluorescence, but spectroscopic and electrical conductance methods are also frequently used. One difficulty with all these detection methods is that it is not possible to assay many different SSAs in a single physical location (e.g., a single tube). If all the SSAs have the same readout (change in fluorescence, change in conductance etc.) it is impossible to tell which SSA in a complex pool has bound its ligand, only that some ligand SSA interaction has occurred. For this reason, all current methods for the detection of SSA ligand binding require different SSAs to be physically separated such as on an array. This necessitates relatively large sample volumes, which may work in situations where material is not limiting, and such arrays can be used to detect the binding of many different ligands to their cognate SSAs in parallel. However, this creates significant problems when sample volumes are extremely small, and this is a major hurdle for single cell analysis. Typical human cells have a volume of ˜1000 fl, which is many orders of magnitude below that needed for typical array platforms. Very small sample volumes and their correspondingly low levels of analyte are not a problem for the detection of DNA or RNA since these can be amplified trivially, which allows for efficient methods to measure gene expression or to sequence entire genomes at the single cell level. However, unlike PCR, there is no analogous method to amplify small molecules such as metabolites. SSAs thus cannot be used for the detection of many different ligands at the single cell level without some way to read out the ligand binding to each SSA independently in the same small volume.

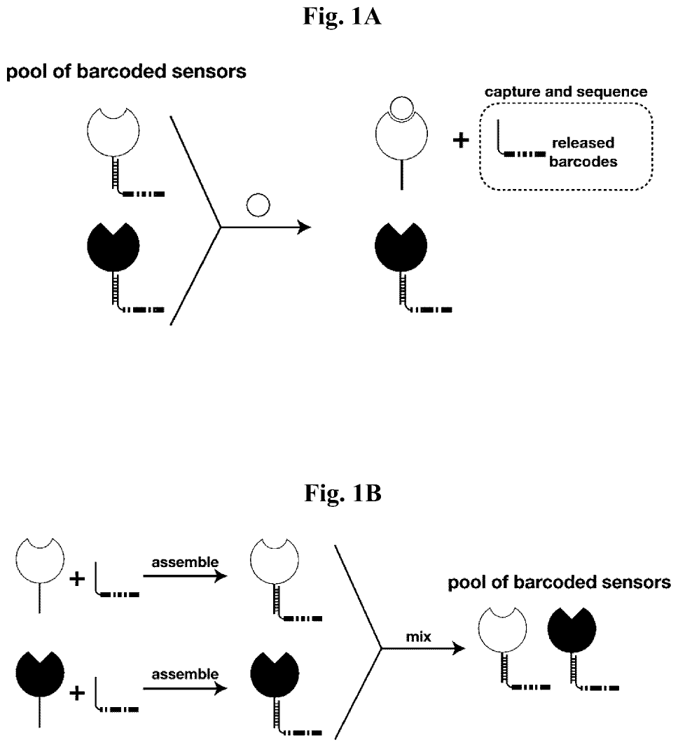

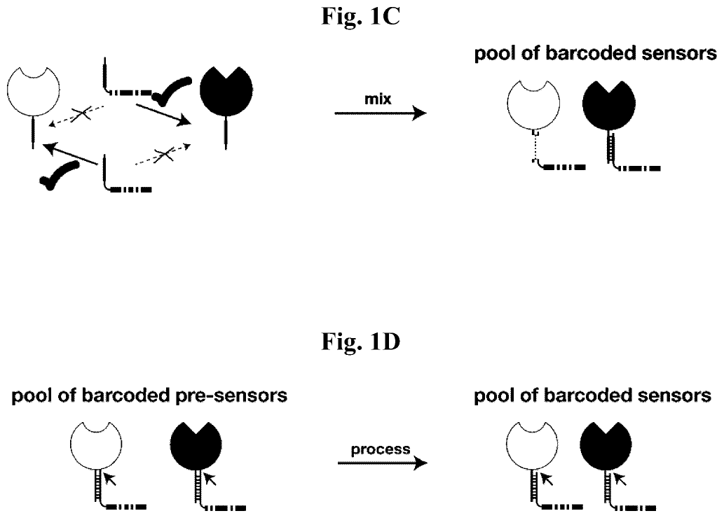

One possible method for the detection of the dissociation of the LBO-SRO hybrid following ligand binding capturing and sequencing of the released barcode region. An SRO or LBO can be engineered to contain a unique short DNA sequence or ‘barcode’ and reading that barcode uniquely identifies which SROs or LBOs have been released through the binding of its cognate ligand. If SSAs for different targets each release SROs with different unique barcodes, then by capturing and sequencing the released SROs or LBOs, it is possible to tell which SSA recognized its target. Thus, barcode sequencing can read out which ligands were present in the sample. Since it is possible to generate many unique barcodes (e.g., a barcode that is 12 bases long can have ˜17 million unique iterations), and each is an independent readout, this allows the parallel measurement of many ligands simultaneously in the same sample. This approach is broadly shown in FIG. 1A and would allow single-cell metabolomics since many thousands of SSAs can be monitored in the same sample volume and barcodes can be amplified trivially using standard nucleic acid amplification methods. The critical hurdle in this approach is not the ability to make functional SSAs, but to correctly pair each individual LBO to its barcoded SRO.

Conceptually, there are several ways to correctly pair different LBOs to unique barcoded SROs. The simplest is shown in FIG. 1B. Every LBO-SRO pair interacts by base-pairing via the same universal sequence and the individual LBO-SRO pairings are done one at a time in separate hybridization reactions. This clearly can work but is not a pragmatic approach for large SSA collections. For example, making a library of SSAs that can cover all possible 10-mer sequences in the ligand binding region of the LBO would require over 1,000,000 individual hybridizations. An alternative method is shown in FIG. 1C. Here, each LBO-SRO pair interacts via a different base-pairing sequence. Conceptually, a large library of LBOs could be mixed with a large library of SROs and the correct pairing would arise through the unique base-pairing between each LBO-SRO pair. To allow this to work, the unique base-pairing regions of each LBO-SRO pair would have to have very similar hybridization characteristics (e.g., a very similar Tₘ). This greatly limits the available sequence space. Every unique base-pairing sequence would also have to all work equally well at responding to ligand binding by driving dissociation—secondary structures and other constraints further limit the possible sequences. Finally, and most problematically, the base pairing of each SRO to its matched LBO via their unique hybridization region has to be error-free. If SROs frequently associate with incorrect LBOs then there will be no way to deconvolute since there is no longer a known match of barcode SRO to LBO and hence no longer a known match between barcode and bound ligand.

Here, in some embodiments, we propose a third alternative as shown schematically in FIG. 1D in which the SSAs are synthesized as unimolecular pre-SSAs—the LBO and barcoded SRO are synthesized together as a single molecule. The barcode is thus implicitly matched to the LBO. The SRO and LBO regions of the pre-SSA associate via a universal base-pairing sequence—every SRO-LBO pair thus associates via an identical sequence and thus all have identical release properties. To convert the unimolecular pre-SSA to the mature bimolecular SSA, we introduce a nick immediately preceding the base-paired SRO-LBO region. This can be done in a variety of ways including cleavage at an abasic site, cleavage at a photocleavable base, or via enzymatic cleavage using a restriction enzyme. This enables us to make libraries of barcoded SSAs of arbitrary size that all have identical association and dissociation properties, since every LBO-SRO pair interacts via the same base-pairing sequence and the exact pairing of each barcoded SRO to its LBO is perfectly known.

### Example 3: Methods for Detecting Ligand Binding Using Barcode SSAs

To generate SSAs that allow ligand binding to be monitored by the release of barcoded SROs, we designed SSAs as shown in FIG. 2B. We note that this SSA design is partly derived from that used by Yang et al., 2014, and other groups (FIG. 2A), however a key difference here is the release of SROs that are used to read out ligand binding, and this method can be used to read out SROs that have been released from a multiplexed pool of SSAs. Since each SRO is a DNA molecule with a unique barcode, it is possible to read out ligand binding to a large library of SSAs in parallel since each SSA has a unique barcode SRO readout. Furthermore, since the binding of a ligand to each SSA can be detected by release of a barcode SRO made of DNA, the ligand binding region of the SSA can be composed of DNA, RNA, any non-natural XNA, other biopolymer, or any combination thereof, since there is no requirement to read the LBO itself. The barcode SRO detection method thus permits both the detection of many SSAs in parallel and the expansion of the potential biochemical composition of the ligand binding region of the SSA.

To test the feasibility of our method for assembling barcoded SSAs, we adapted an SSA architecture from Yang et al., 2014, though we note that many other stem sequences and precise architectures are also possible. This is shown in FIG. 2A and was the basis for SELEX experiments to identify aptamers that recognized the aromatic amino acids tryptophan (Trp), phenylalanine (Phe), and tyrosine (Tyr). In broad outline, a short universal oligo is fixed to a solid matrix and this hybridizes to the ligand binding oligo (LBO). Ligand binding drives a conformational change in which a unimolecular base-pairing event disrupts the base-pairing between the short-bound oligo and the LBO, resulting in the dissociation of the LBO and its release from the solid support. In one embodiment, we flipped this architecture so that the LBO is now linked to a solid matrix via its 5′ end—using a 5′ biotin group that binds tightly to streptavidin on the surface of magnetic beads. In this altered configuration, the short oligo is now released following ligand binding (FIG. 2B). This should in principle allow a large library of different LBO-SRO pairs to all be bound to the same solid matrix (FIG. 2C) and only the SSAs that bind their cognate ligand release their barcoded SRO. The remainder of the barcoded SROs remain associated with their LBOs, and these can all be removed by removing the magnetic beads.

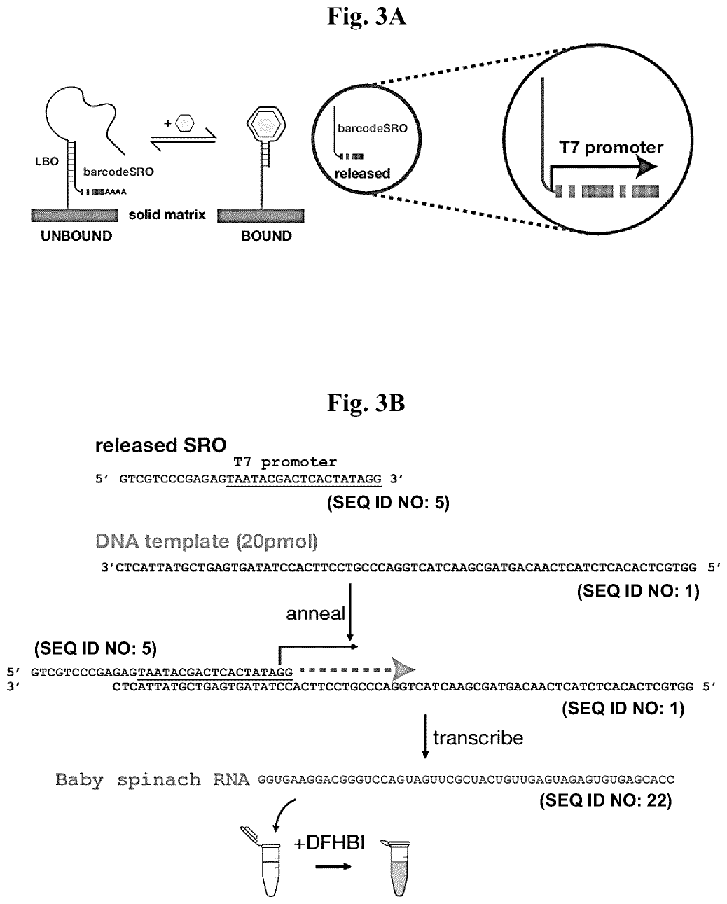

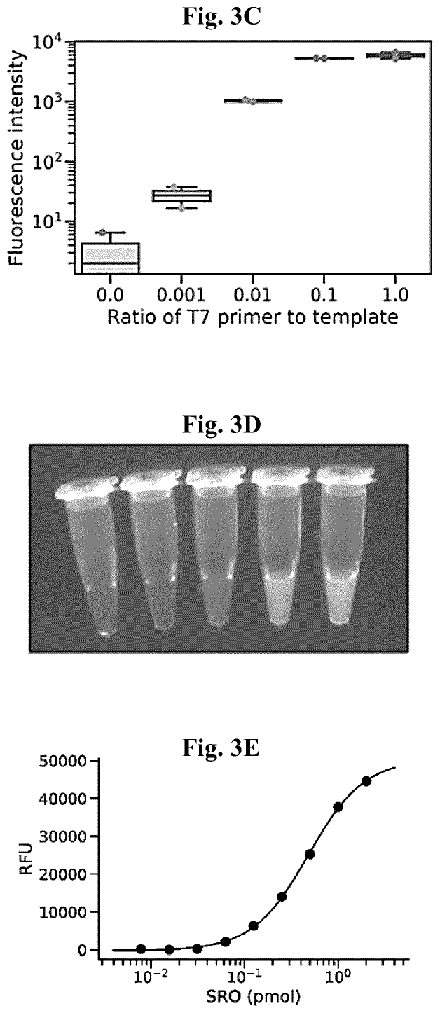

To test whether the architecture works, we first established a simple assay to detect released barcode SRO. While we could of course use sequencing to count the number of molecules of released SRO, we wanted a simple and immediate readout of barcode release and thus, we established the method shown in FIG. 3. Instead of using an arbitrary sequence barcode attached to our SRO, we chose a barcode that corresponds to the T7 promoter (FIG. 3A). This allows us to detect barcode SRO release with a simple fluorescent reporter. In brief, the released T7 barcode SRO hybridizes to a second oligo, which is the reporter template. The region of the T7 promoter is now double-stranded, which is essential for it to act as a promoter for transcription (FIG. 3B). The reporter is a sequence that produces the Baby Spinach RNA when transcribed, which is a 51-nt RNA that fluoresces when it binds the molecule DFHBI. In this way, SSA dissociation can be monitored simply by fluorescence. As shown in FIGS. 3C-3E, titration of the T7 barcode into a reaction containing excess Spinach reporter results in dose-dependent fluorescence over at least 3 orders of magnitude, allowing us to monitor SSA ligand binding efficiently. We note that this assay could also work with any other promoter elements (e.g., T3, SP6) and any fluorescent or functional RNAs (e.g., ribozymes) and the released barcode SRO could be used as a primer for reaction including but not limited to PCR.

### Example 4: Barcode Release can be Used to Detect Ligand Binding

We adapted the tryptophan (Trp) SSA reported in Yang et al., 2014. The version they report has an LBO release architecture as shown in FIG. 2A—we adapted this to our new barcode SSA release architecture and use the T7 promoter as the barcode to allow us to use our fluorescent reporter assay. To test whether the architecture works, we hybridized our biotinylated LBO to our T7-barcoded SRO and bound the bimolecular assembly onto streptavidin-coated magnetic beads (FIG. 4A). We either added 10 μM Trp, 10 μM leucine (Leu), or 10 μM Phe to drive SRO release, then removed any remaining SRO-LBO SSA with unreleased SRO by removing the magnetic beads. We then ran our T7 reporter transcription reaction on the supernatant, added DFHBI-1T, and measured fluorescence. We also resuspended the beads, boiled them to dissociate any remaining barcoded SRO, and ran an identical T7 reporter transcription on the SRO released from the beads. This allows us to determine what proportion of initial bound SRO was released due to ligand addition. The results are shown in FIG. 4B. Addition of Trp results in successful release of barcoded SRO from the SSA whereas neither Leu nor Phe resulted in significant release. This shows that barcode release can report on binding of a ligand to an SSA.

To confirm that this was not limited only to the Trp SSA, we repeated an analogous set of experiments with the Phe and Tyr SSAs described in Yang et al., 2014. We adapted their Phe and Tyr SSA sequences to our barcode SRO release architecture exactly as was done for the Trp SSA. We found that Phe and Tyr drove efficient barcode SRO release from their respective SSAs (FIG. 4C). In addition, like the Trp SSA, we show this release to be specific to their respective ligands, with minimal SRO release either in the presence of 10 μM of Leu or 10 μM of other aromatic amino acids (FIG. 4D). With the Phe SSA, we also added an additional negative control—we used a scrambled LBO where the region of the LBO that is thought to interact with the Phe has an identical nucleotide content as the true Phe SSA but the sequence is scrambled. This should not bind Phe. We added 100 μM Phe to either the real SSA or to the scrambled control and repeated the T7 reporter assay as before. We found that while Phe drove efficient barcode SRO release from the SSA with the correct ligand binding region (FIG. 4C) it failed to drive any detectable release from the scrambled ligand binding region control (FIG. 4E). In addition, we added different doses of ligand to the Phe and Tyr SSAs, with EC₅₀ values in the low μM range (FIG. 4F).

We also showed that our SRO release architecture works with other classes of ligands by adapting SSAs originally reported by Coonahan et al., 2021 that bind the anti-malarial drugs piperaquine (PQ) and mefloquine (MQ). Using the same barcode SRO release method, we showed that addition of piperaquine (FIG. 4G), mefloquine (FIG. 4H), or cortisol (FIG. 4I) (reported in Yang et al., 2017) results in successful release of barcoded SROs from the SSAs in a dose-dependent manner.

### Example 5: Barcode Release can be Used to Detect Ligand Binding in Multiplex Assays with Negligible Crosstalk

The experiments in Example 4 and in FIG. 4 show that barcode SRO release can be used to measure ligand binding to an SSA. However, when we compared the release of the barcode SRO between the true SSA and the control with the scrambled LBO ligand binding sequence, these were done in separate assay tubes. In a true multiplex experiment, every SSA would be in the same tube, in the same assay volume. There could potentially be crosstalk between SSAs such that ligand binding to one SSA could displace the SRO from a separate unrelated SSA. To test this, we first mixed the Phe SSA and the scrambled Phe SSA control in the same tube and assayed barcode SRO release in two different ways. In the first version, the true Phe SSA had an SRO barcoded with the T7 promoter whereas the scrambled version had an SRO barcoded with the T3 promoter (FIG. 5A). Any T7 polymerase driven transcription of the Baby Spinach reporter (and hence any fluorescence) must come from the true Phe SSA. In the second tube, we reversed the barcodes—in this case the scrambled SSA had the T7 barcoded SRO and the true Phe SSA had the T3 barcoded SRO (FIG. 5B). Any fluorescence observed in our T7 reporter reaction in this control assay must have been due to release of barcoded SRO from the scrambled Phe SSA. Since we detected no such release when we assay this scrambled SSA alone (FIG. 5B), any observed release must be coming from cross talk from the true Phe SSA. We observe strong fluorescence in the assay where only the true Phe SSA has a T7 barcoded SRO (FIG. 5A) and observe negligible fluorescence in the assay where only the scrambled Phe SSA has the T7 barcoded SSA (FIG. 5B).

In a second test, we again mixed two SSAs in the same tube—but this time with two SSAs that recognize different ligands. We mixed the piperaquine SSA and mefloquine SSA in the same tube in a similar setup as above. In the first tube, the piperaquine SSA had an SRO barcoded with the T7 promoter whereas the mefloquine SSA had an SRO barcoded with the T3 promoter (FIG. 5C). In the second tube, the barcodes were reversed and the mefloquine SSA had the T7 barcoded SRO and the piperaquine SSA had the T3 barcoded SRO (FIG. 5D). We then added either 50 μM piperaquine or mefloquine to the mixed pool of SSAs. As expected, we observe strong fluorescence only upon piperaquine addition in the assay where the piperaquine SSA had a T7 barcoded SRO (FIG. 5C). In contrast, in the assay where the mefloquine SSA had the T7 barcoded SSA we observe strong fluorescence only upon mefloquine addition (FIG. 5D). We therefore conclude that there is negligible crosstalk between SSA molecules in the same reaction using our architecture.

The data to this point shows that we can take an SSA identified by traditional SELEX or other conventional means to bind a specific amino acid (Trp, Phe or Tyr), drug (PQ, MQ), or hormone (cortisol), change the configuration, and convert it into an SSA that can give barcode SRO release as a specific readout of ligand binding. In addition, we show that multiple SSAs can be assayed together in the same assay volume without cross talk—the released barcodes are specific to the SSA that binds the ligand.

In a third test, we again mixed two SSAs that recognize two different ligands (cortisol and piperaquine) in the same tube, except that the different SSAs were associated with SROs attached to different fluorophores instead of barcodes (FIG. 6A). The SRO hybridized to the piperaquine (PQ)-binding LBO was conjugated to a green fluorescent probe (6-FAM), while the SRO hybridized to the cortisol-binding LBO was conjugated to a red fluorescent probe (TEX615). This allowed us to measure the release of both sets of SROs simultaneously by detecting fluorescence signals in two different channels (red and green). The two SSAs bound to beads were mixed in equal amounts then incubated with either 50 μM of PQ (FIG. 6B) or 200 μM of cortisol (FIG. 6C) before the percentage release of their respective SROs was evaluated by measuring fluorescence in the red and green channels. Incubation with PQ or cortisol resulted in expected increases in green (PQ; FIG. 6B) and red (cortisol; FIG. 6C) fluorescence signals, respectively. Interestingly, approximately 20% non-specific SRO release was observed in the absence of ligand for the cortisol SSA in FIG. 6B and the PQ SSA in FIG. 6C, which is a background significantly higher than that was observed with the barcoded SROs employed in FIGS. 5A to 5D.

### Example 6: Converting Non-SSA Aptamers into a Barcode SSA

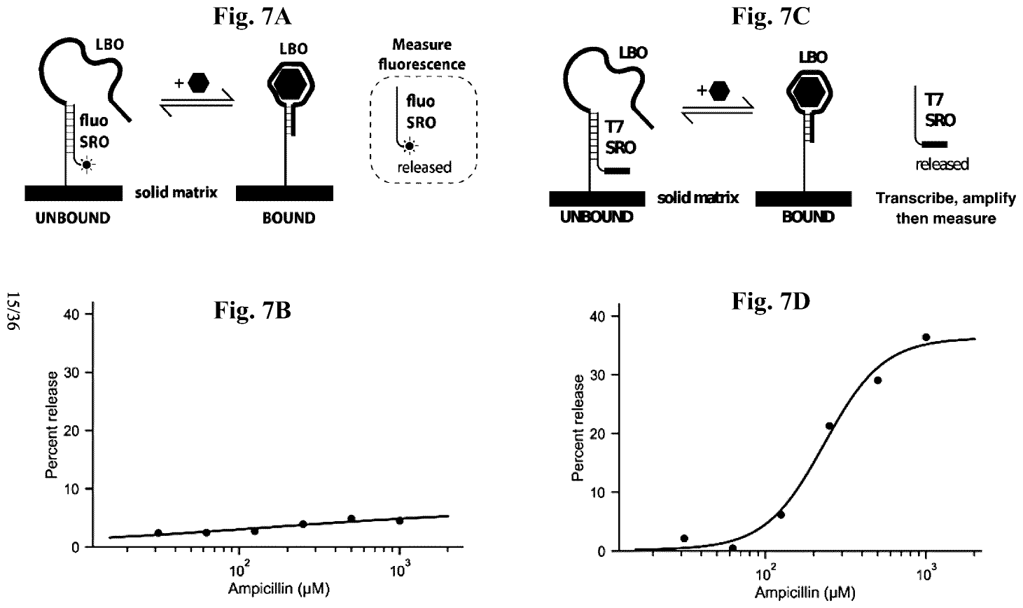

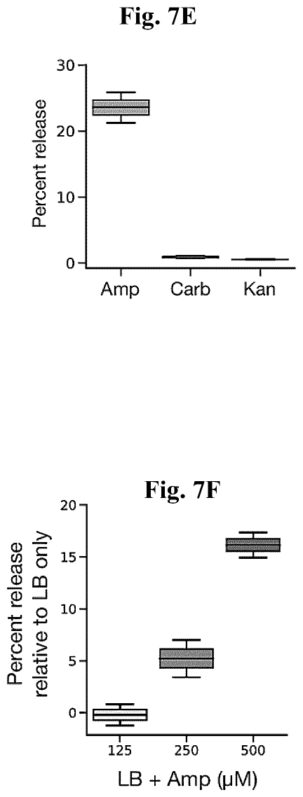

The SSAs we assayed in FIG. 4 above had been isolated specifically to work as SSAs. However, there are many other ligand binding aptamers that were not identified as SSAs, but simply identified via SELEX for their ability to bind a known ligand. We wanted to explore whether it is possible to convert one of these non-SSAs into a barcode-releasing SSA. We thus selected the ampicillin-binding aptamer AMP17 that had previously been identified by Song et al., 2012 as a ligand binding non-SSA. We initially tested the AMP17 aptamer with an SRO labeled with the fluorophore 6-FAM (6-carboxyfluorescein; MW: 376; FIG. 7A) and detected less than 10% SRO release (FIG. 7B). We then constructed and tested the AMP17 aptamer with an SRO containing a T7 SRO (using the same starting amount of DNA) and measured release using our T7-Baby Spinach assay (FIG. 7C). Interestingly, we detected significantly more SRO release when the T7 SRO comprising the bulkier T7 promoter sequence (MW: 5874) was used (FIG. 7D). In addition, we found that the T7 SRO release was specific to ampicillin, as minimal release was detected in the presence of other antibiotics such as kanamycin or the ampicillin analog carbenicillin (FIG. 7E). This shows that the ability of our SSA architecture to allow the construction of barcode release SSAs does not require the ligand binding region to have been identified initially as an SSA but is more widely applicable. Furthermore, we also showed that our ampicillin barcode SSA can detect ampicillin in the context of a complex mixture of ligands. We incubated our ampicillin SSA in LB medium containing different amounts of ampicillin and find that we can detect ampicillin specifically in the context of a complex soup of small and large molecules including peptides, carbohydrates, and amino acids (FIG. 7F).

### Example 7: Using Barcode SSAs to Detect Small Molecules in Complex Mixtures

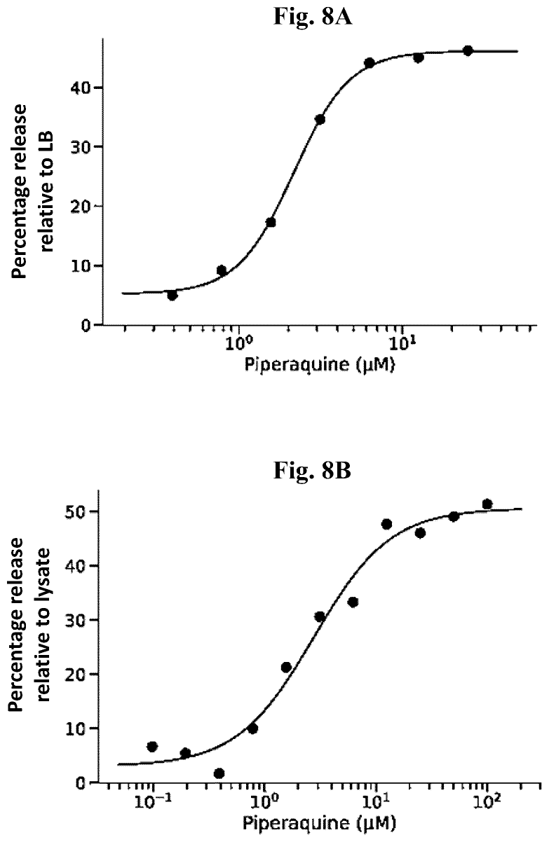

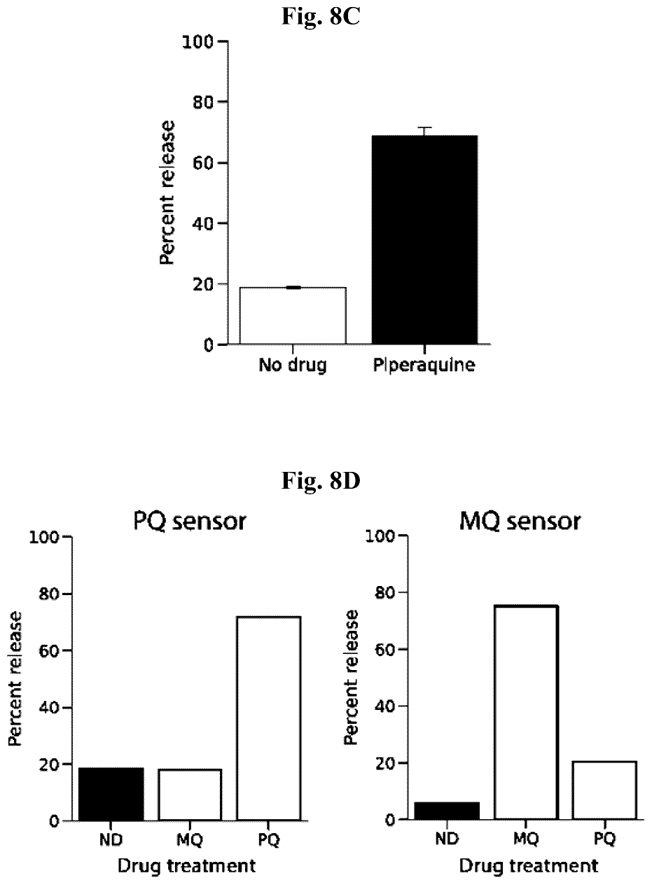

To further test that our barcode release method works in the context of complex mixtures of biomolecules, we also incubated the piperaquine SSA in LB medium spiked with different amounts of piperaquine and found that, like the Amp SSA, the piperaquine SSA can detect piperaquine specifically in this complex mixture (FIG. 8A). In addition to detection in LB, the piperaquine SSA also shows a similar dose-dependent response in cell lysate derived from whole C. elegans worms (FIG. 8B). Next, to test if our sensors can also detect drug uptake in whole animals, we treated worms with either no drug, piperaquine or mefloquine for 7 h. We then washed away the drug medium, lysed the worms, and then incubated the lysate with the barcoded SSAs. The piperaquine and mefloquine SSAs were able to specifically detect their cognate ligands in the lysate of worms treated with piperaquine and mefloquine respectively (FIGS. 8C and 8D). Our barcoded SSAs should thus be able to detect their correct ligands in biologically relevant fluids such as plasma, tears, cell culture medium, or in the case of single cell assays, cytoplasm.

### Example 8: Converting a Unimolecular Pre-SSA to a Mature SSA Results in an Active SSA with Barcode Release as Readout

All the experiments described above were using SSAs produced by hybridizing an LBO to an SRO to produce the SSA and then monitoring ligand binding by monitoring barcode release through our T7 barcode release fluorescence assay. The experiments showed that our T7 fluorescence assay provides an easy and rapid assay for barcode release (FIG. 3) and that this accurately reads out ligand binding either to aptamers isolated as functional SSAs (FIG. 4) or by adapting aptamers isolated purely through substrate affinity (FIG. 6). We have thus shown that barcode release is an excellent method to readout ligand binding to an SSA. To make this feasible in large scale collections of SSAs, or even in full SSA libraries, we need an efficient way to produce the mature barcoded SSAs such that we can guarantee that each barcoded SRO is correctly paired to its corresponding LBO.

As shown in FIG. 1, there are two efficient ways to do this. The first is to have a unique base-pairing sequence between each barcode SRO and its LBO. A pool of SROs and LBOs could thus be mixed and in theory it might be possible for each SRO to hybridize uniquely to its correct LBO partner. This is difficult and the sequence space for the hybridizing region is highly restricted since these different base-pairing regions must all have very similar sequence composition to ensure similar hybridization and release properties. In addition, the hybridizing of SRO to LBO in the large mixed pool must be error free otherwise incorrect SRO-LBO pairs can form making deconvolution very challenging. Finally, different hybridizing regions of the same sequence composition but different sequence order should all be functional and be able to make effective SSAs. This method is unlikely to be suitable for large multiplex applications, given the small sequence space, the challenge of perfect hybridization between only the desired SRO-LBO pairs in complex pools of oligos and due to the unknown requirements for the hybridization sequence. Nonetheless, to partly test the feasibility, we tested the last question: do all sequences of the same sequence composition but different sequence order behave the same as hybridization regions of our SSAs?

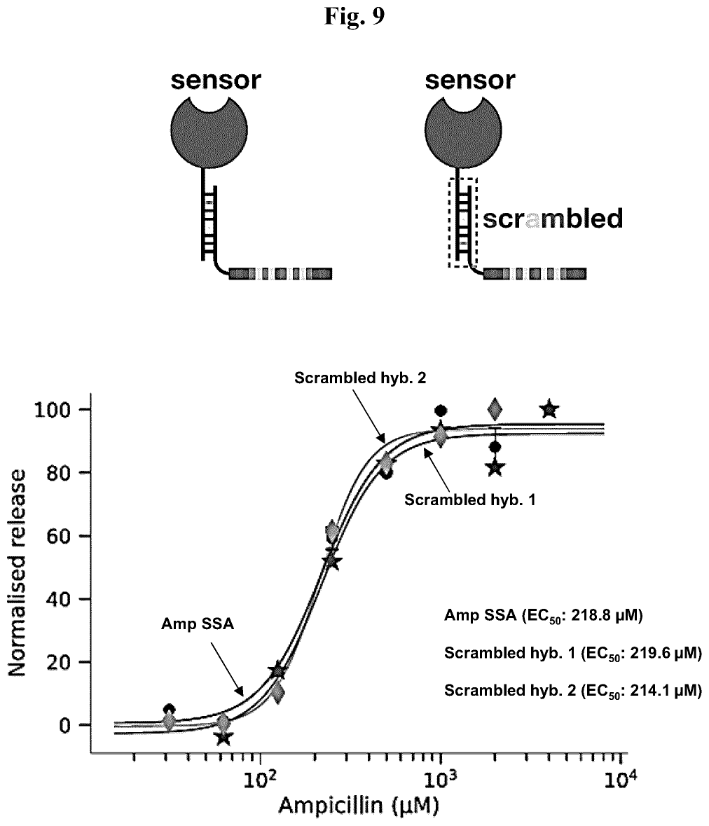

We generated SSAs where the ligand binding region is the same, but the hybridization region has been scrambled. Note that the intramolecular complementarity is preserved, therefore this scrambled hybridization region should be able to bind to the complementary scrambled SRO and should be able to associate with itself just as with the normal hybridization region. We show that we can obtain ligand-mediated SRO release with these scrambled hybridization sequences, such as with the ampicillin SSA, and that the dose response curves obtained are very similar (FIG. 9).

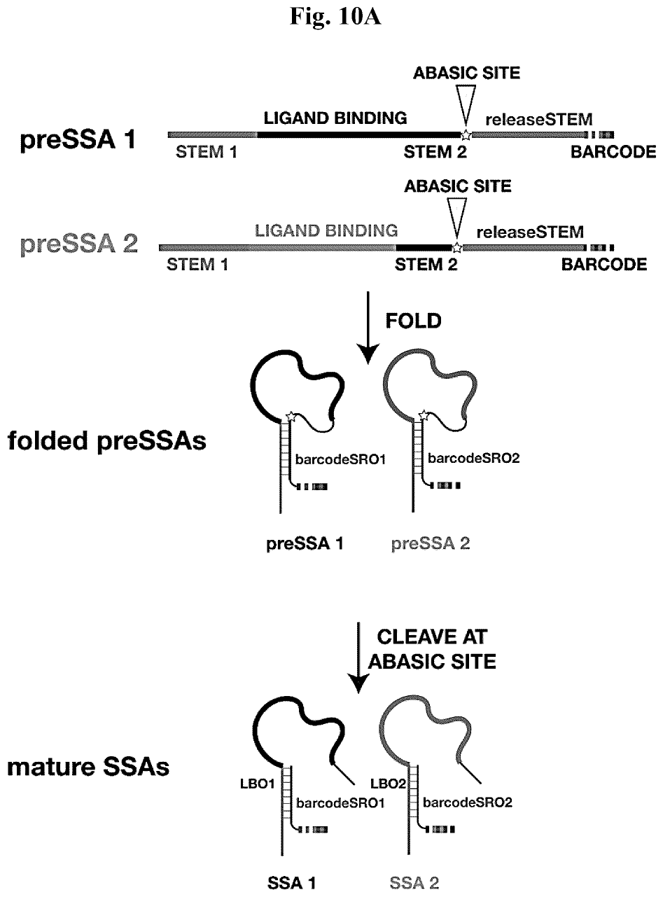

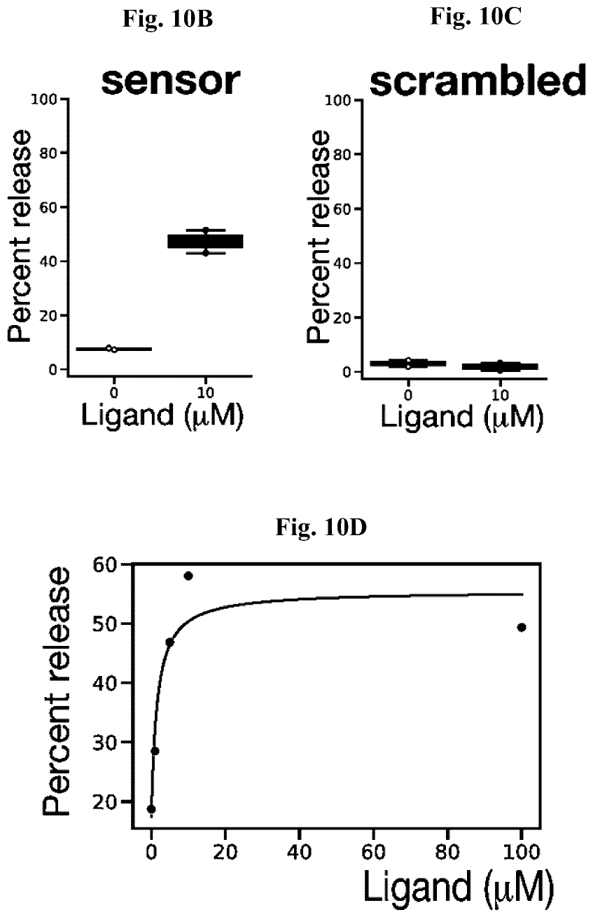

In the context of library construction, while we should in principle be able to use SSAs with different hybridization sequences to construct our SSA libraries as depicted in FIG. 1C, we expect there to be small differences in SSA behaviour that may complicate library assembly. For this method to work efficiently, we would have to design the sequences to have similar hybridization characteristics (e.g., Tₘ) and release parameters, which would be time-consuming. We thus turn to testing the alternative method that we believe is more easily workable (FIG. 10).

In this method (see FIG. 10A for schematic), SRO and LBO are synthesized in a single initial molecule—we term this a pre-SSA. Since every LBO and SRO pair are made as a single pre-SSA molecule, the SRO and LBO pairing is implicit. We allow this molecule to fold so that the SRO and LBO regions pair using the hybridization region—note that this region is identical for every pair of SRO and LBO and thus every SSA has the same hybridization and release properties. Finally, we nick the pre-SSA, converting the unimolecular pre-SSA into its mature bimolecular form. We do this in the examples shown here by incorporating an abasic site at the exact point where we want to cleave, but there are multiple other alternatives such as using a photocleavable site or a restriction enzyme produced nick.

To test this, we synthesized a pre-SSA containing an abasic site to generate a mature SSA with an LBO corresponding to the Trp SSA reported in Yang et al., 2014 and a T7 barcoded SRO (preSSA 1; FIG. 10A). We also generated a control pre-SSA to generate a mature SSA with a scrambled version of the ligand binding region of the Trp LBO—this should not be able to bind Trp (preSSA 2; FIG. 10A). We folded the pre-SSAs and nicked them by cleaving at the abasic site to generate the mature SSAs. We added Trp to either the real Trp SSA or the scrambled version and assayed barcode SRO release using our T7 transcription of Baby Spinach fluorescence assay. We find that the real Trp SSA releases the barcoded SRO efficiently (FIG. 10B) whereas the scrambled ligand binding region version shows negligible release (FIG. 10C). Finally, the mature Trp SSA shows a very similar dose response to that reported by Yang et al., 2014 for their SSA which has an identical ligand binding region but different architecture and a completely different readout (FIG. 10D). We conclude that we can make mature functional SSAs with known SRO-LBO pairings by taking unimolecular pre-SSAs and converting them to mature SSAs. This can be done for libraries of any arbitrary size and allows us to efficiently produce large collections of SSAs where ligand binding can be monitored by assessing the release of barcoded SROs.

### Example 9: Increasing the Chemical Space of the Ligand Bind Region

Most SSAs have been identified by a screening system. A massive library of SSAs is synthesized where each SSA has a different ligand binding region in the LBO and every SSA has the exact same SRO. The ligand binding region is typically long, e.g., a 50mer or 60mer. While this provides a massive sequence space out of which to select the high affinity SSAs, this method imposes a major limitation on the chemistry of the SSA ligand binding region.

The complexity of the starting oligo pool is so vast that each of sequence is present at most once in the aliquot of the library being screened in a given SSA SELEX experiment. There must therefore be many rounds of amplification. This need for amplification imposes major limitations on the nucleic acid chemistry used in the SSAs since each round of amplification requires enzymes including polymerases and reverse transcriptases. These enzymes can only use RNA or DNA as templates—the entire of conventional SELEX is thus restricted to these conventional nucleic acids. This excludes xeno nucleic acids (XNAs). XNAs can be chemically extremely diverse—while they share the same basic backbone as RNA and DNA instead of using ribose or deoxyribose sugars they can have a much greater variety of side groups. These all have different chemical properties and will allow different binding capabilities. In conventional SELEX, the template needs to be amplified by polymerases in multiple cycles. However, XNAs can typically not be read or synthesised by conventional polymerases, and it requires extensive engineering to generate polymerases capable of incorporate specific XNA bases, greatly limiting the repertoire of possible XNAs that can be used. In addition, while one engineered polymerase can incorporate one type of XNA, it often fails with other XNAs—thus it is currently almost impossible to select ligand binding regions that comprise a mixture of different classes of XNAs. Finally, nucleic acid polymerases do not synthesise other types of polymers such as peptides, lipids, and polysaccharides. There are thus major limitations in the composition of any SSA selected by SELEX.

Our design of barcode SSAs allows the use of any XNA or indeed any other type of polymeric organic molecule (e.g., peptide) that can be inserted into the ligand binding region of the LBO. The main restriction is on the chemistry of the SRO and the region it base pairs with on the LBO. As long as the SRO that is released is either RNA or DNA, that can be ‘read’ with conventional enzymes. This opens the possibility of using a range of chemistries in our ligand binding region, including having a mixture of different chemistries in the same ligand binding region.

### Example 10: Increasing the Physical Size of the Ligand Bind Region while Maintaining Defined Complexity

The ligand binding region of the LBO in conventional SSA SELEX is long, e.g., 30mers, 50mers or 60mers are often used. This allows for a large ligand binding loop to incorporate larger biomolecules. For example, glucose or serine are relatively small (<1 nm in diameter). A 10mer oligo is ˜3.4 nm in length—in a loop configuration this gives a loop of ˜1 nm in diameter, sufficient to bind glucose or small amino acids. However, larger molecules like ATP or NAD cannot fit into a 10mer pocket and require longer ligand binding regions e.g., a 30mer has ˜3 nm diameter sufficient to bind ATP, NAD or even small phospholipids. The problem in conventional SSA ligand binding region design is a trade-off: the longer the ligand binding region, the larger the molecules it can bind but also the greater the complexity of the library and the greater need for amplification and the more poorly defined the starting SSA pool is. For example, in Yang et al., 2014, they start with a library with a N30 complexity in the ligand binding region—this would therefore allow binding to larger molecules like ATP. However, this gives ˜1.2E+18 possible sequences. Each selection starts with 0.15 nmoles of the library—they are therefore screening only 1 in 10,000 of all possible sequences and they do not know what subset they are screening.

Here we take an approach of controlled complexity libraries where the maximum number of variable positions is 12—the total library size is thus ˜17 million molecules. Note that a single magnetic bead such as a Dynabead™ has 10 million streptavidin molecules on its surface. If we screen 0.1 nmoles of a N12 library (a similar number to that in Yang et al., 2014, each sequence would be represented over several million times.

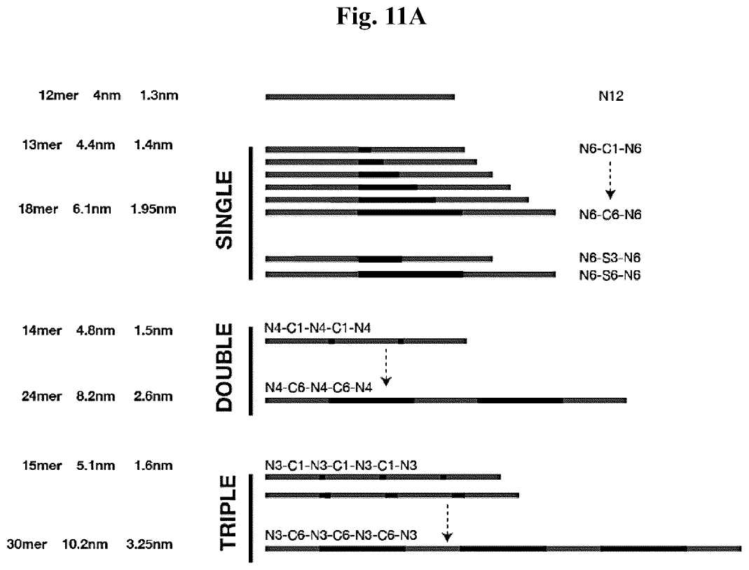

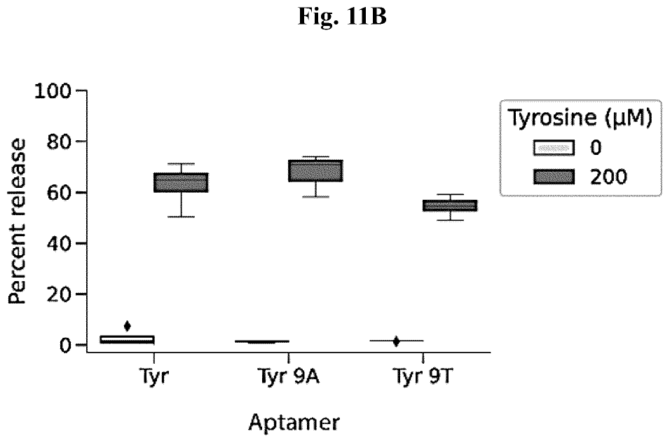

In its most basic form, a controlled complexity N12 library gives a length of ˜4 nm or a diameter of loop of 1.3 nm. However, as shown in FIG. 11A, we expand the dimensions of the ligand binding region by adding constant elements in different geometries. These can be short regions of fixed nucleotide sequences or insertions of other chemistries such as polyethylene glycol spacers or insertions of fixed secondary structures such as hairpin loops. This allows us to expand the physical dimensions of the loop without altering the sequence complexity of the library. We called these controlled complexity libraries. To show that we can add short fixed sequences to the ligand-binding loop without affecting ligand-specific contacts, we adapted the tyrosine aptamer from Yang et al., 2014 to our barcode architecture. We kept the 10 nt of consensus sequence identified by Yang et al., 2014 but replaced the 7 non-consensus bases in the ligand-binding loop with a sequence of 9 adenine or 9 thymine bases. Using our barcode release method, we show that the SSAs with the A or T replacements respond to tyrosine in a similar manner as the original tyrosine sequence (FIG. 11B). This suggests we can add fixed spacers to expand the loop dimensions without increasing the sequence complexity of our libraries.

### Example 11: Unique Barcode Release and Amplification can be Used to Detect Ligand Binding

As shown in Example 4, ligand binding was detected via barcode release using our fluorescent T7 reporter assay (FIG. 3B) on a variety of small molecules (FIG. 4). In this configuration, the T7 promoter served as the barcode itself within the SSA architecture.

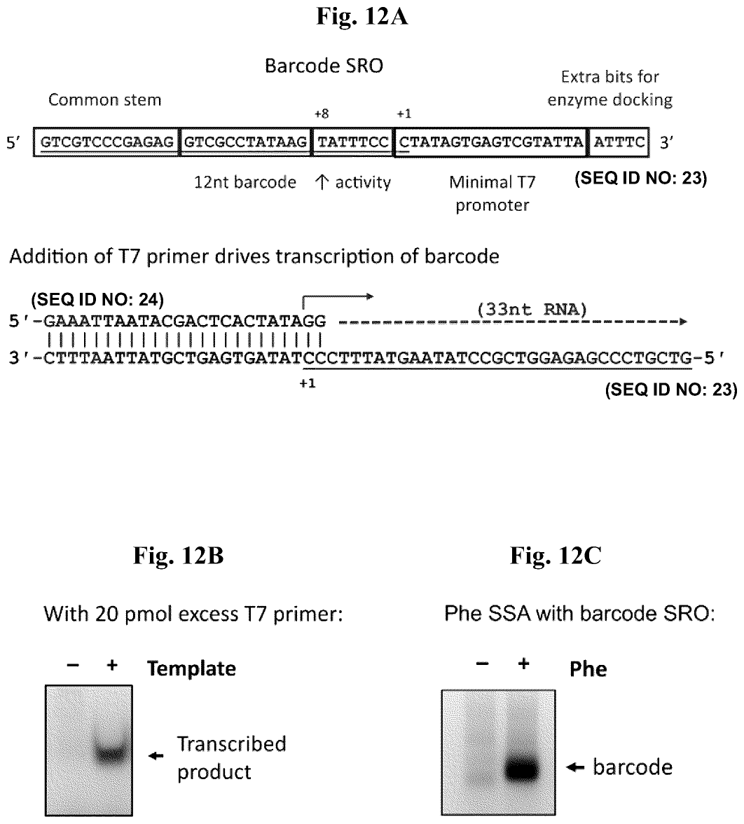

Next, we show that we can incorporate both the T7 promoter and a unique barcode sequence (SEQ ID NO: 23) in the same SRO—having the T7 promoter upstream of the barcode sequence allows for the amplification of the barcodes before sequencing (FIG. 12A). By adding excess T7 primer (SEQ ID NO: 24) to the released SROs, we get transcribed products only in the presence of barcode SROs (FIG. 12B). We further show, using the phenylalanine (Phe) SSA, that the barcode is only amplified upon ligand-mediated SRO release (FIG. 12C).

These data demonstrate that unique barcodes can be released and amplified to detect different ligands within a sample.

### Example 12: Converting a Unimolecular Pre-SSA to a Mature SSA Using a Restriction Enzyme

Next, to facilitate pairing of LBO and SRO, we synthesized them in a single initial molecule named pre-SSA. We allow this molecule to fold to pair the LBO with its corresponding SRO, and we then nick the pre-SSA, converting it into its mature bimolecular form. As described in Example 8, this can be done by incorporating an abasic site at the exact point where cleavage is desired. Other alternatives include using a photocleavable site or a restriction enzyme to produce the nick.

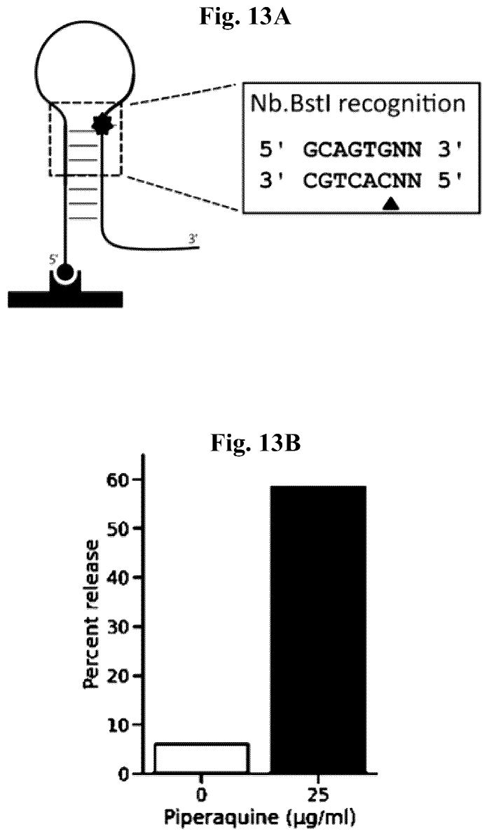

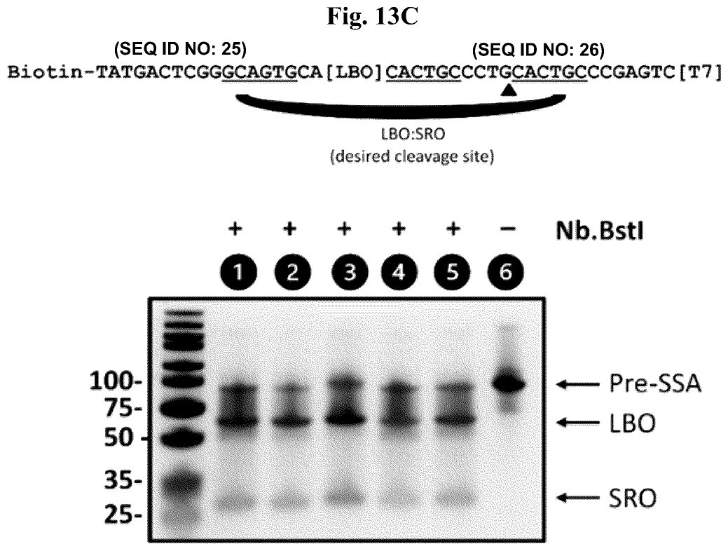

Here, we show that a restriction enzyme, in particular a nicking endonuclease that only cleaves one strand of a dsDNA substrate, cleaves the pre-SSA into its biomolecular form. Nb.BstI was shown to cleave pre-SSAs when its restriction site is incorporated in the common stem sequence of SSAs (FIG. 13A). Of note, changing the stem sequence to add this restriction site does not abolish the function of our SSAs (FIG. 13B) and folded pre-SSAs can be cleaved by Nb.BstI into its bimolecular parts (FIG. 13C), suggesting that our system is flexible enough to be used with different restriction enzymes for pre-SSA processing.

### Example 13: Increasing the Chemical Space of the Ligand Binding Region

Our design of barcode SSAs allows the use of any XNA or any other type of polymeric organic molecule (e.g., peptide) that can be inserted into the ligand binding region of the LBO. As the SRO is the only region that will be read or sequenced, the primary restriction in modulating the SSA chemistry is on the region that the SRO base pairs with on the LBO.

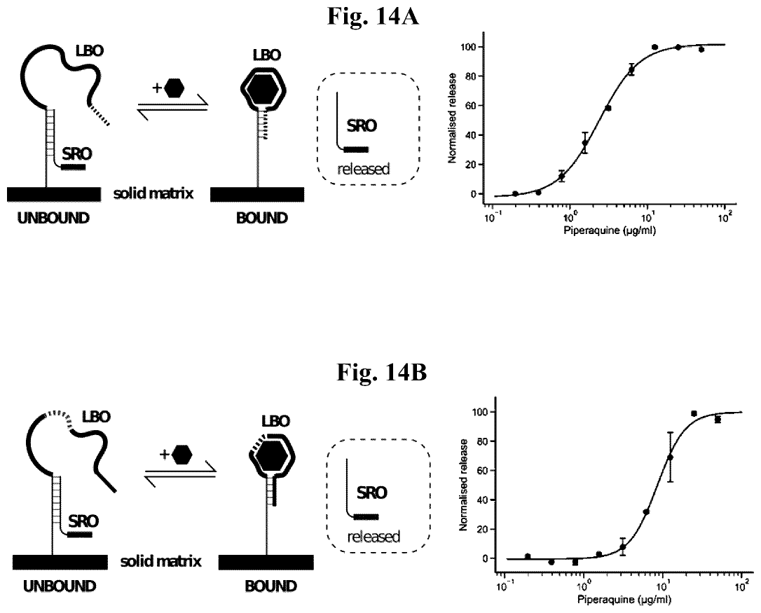

To show that we can have chimeric sequences in the LBO comprising of DNA and non-DNA bases, we substituted regions of the PQ LBO with 2′-O-Methyl-RNA (2′-OMe-RNA). We first substituted the 8 nt region that base pairs with the stem with 2′-OMe-RNA and show that the resulting SSA still functions and responds to its cognate ligand using the same SRO readout (FIG. 14A). Since DNA:RNA duplexes have different melting temperatures relative to DNA:DNA duplexes, we suggest that use of 2′-OMe-RNA (or RNA) bases may be one way to alter and optimize release parameters of the SRO. We next substituted 4 DNA bases in the ligand-binding region with 2′-OMe-RNA bases and show that this construct still functions as an SRO-releasing SSA, albeit with lower ligand-binding affinity (FIG. 14B). These data suggest that we can indeed insert and mix any nucleic acids (or XNAs) in the ligand-binding region of the LBO as the only limit on chemistry is with the SRO.

### Example 14: Selections Using N12 Controlled Complexity Libraries

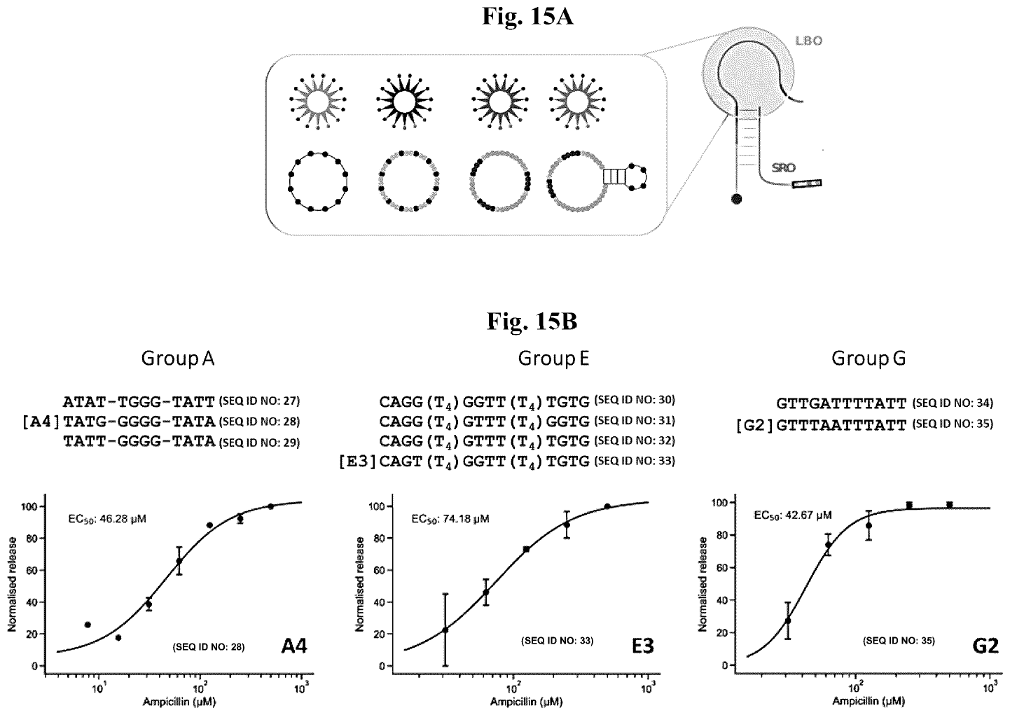

For the selections of new SSAs, we used N12 libraries (SEQ ID NOs: 27-35) that would allow us to sample every possible sequence in the starting pool. In addition to the basic form of an N12 variable region in the ligand-binding region, we also expand the dimensions of the ligand binding region by adding constant elements in different geometries (FIG. 15A). This allows us to expand the physical dimensions of the loop without altering the sequence complexity of the library.

We have since done multiple preliminary selections using a subset of these libraries on beta-lactam antibiotics, including penicillins (ampicillin, carbenicillin, penicillin G, amoxicillin) and cephalosporins (cefaclor, cefalexin), amino acids like tyrosine, and other drugs like piperaquine. We have re-tested some hits from our ampicillin selection and show that we can obtain SSAs using our basic N12 configuration library (Group G), as well as in configurations incorporating fixed nucleotide spacers (e.g., T₄ spacers in Groups A and E) (FIG. 15B).

### Example 15: Alteration in Structural Element Sequences can Alter Dynamic Range of SSAs

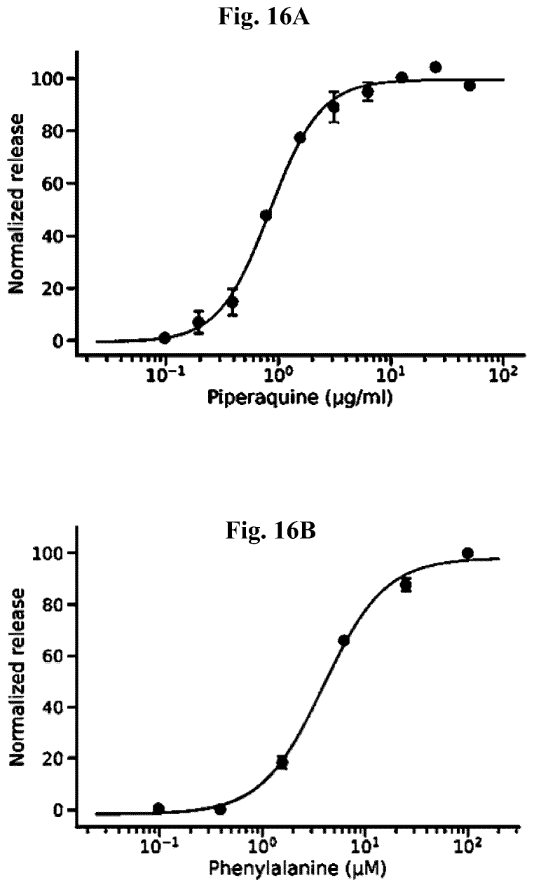

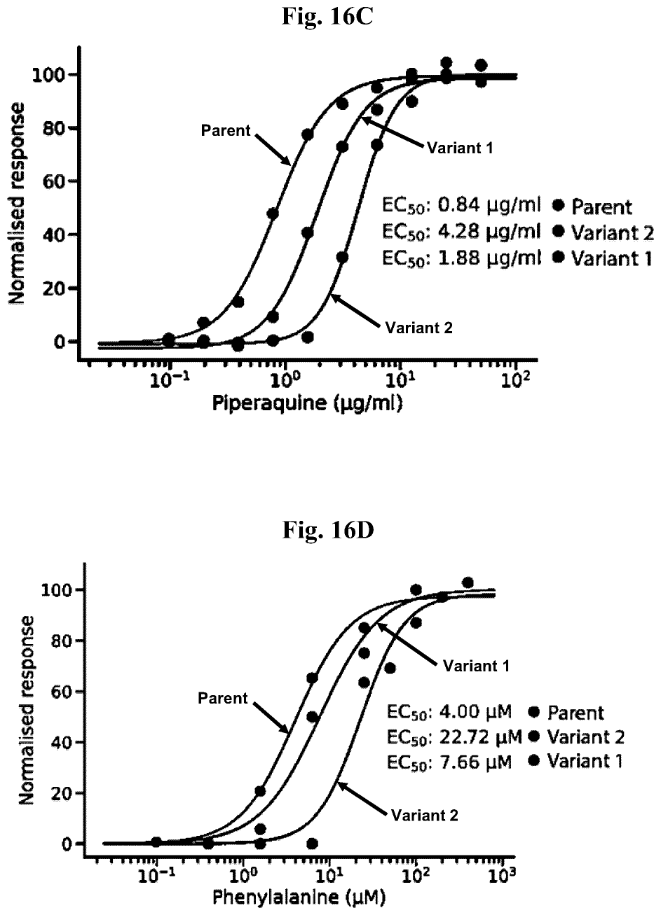

SSAs are powerful and specific but have a key limitation. Each SSA has a defined dynamic range where changes in concentration of its target can be read out as changes in activity of the SSA, as shown for example with the previously used piperaquine and phenylalanine SSAs (FIGS. 16A and 16B). However, if a target concentration lies outside this dynamic range, the SSA is no longer useful as a sensor, either if the desired concentration is below its limits of detection or if we wish to identify differences in concentrations above the linear range.

We therefore wanted to determine whether changing structural elements could alter the response curve of each SSA. We thus made a series of PQ and Phe SSAs where the ligand binding region was unchanged, but the stem sequence was shuffled while maintaining the same base composition and thus, the same binding energy—the altered stem sequences are shown in Table 3.

TABLE 3
Stem sequences used in Example 15

| | | |
|---|---|---|
| | Stem | |
| LBO Sequence | Length | EC₅₀ (μM) |

Phenylalanine

| | | | |
|---|---|---|---|
| Parent | CTCTCGGGACGACGGACGCTAATCTTACAAGGGCGTAGTGTATGTC | 15 | 4.00 |
| | GTCCC (SEQ ID NO: 2) | | |
| Variant 1 | CTCTCGGGCAGACGGACGCTAATCTTACAAGGGCGTAGTGTATGTC | 15 | 7.66 |
| | TGCCC (SEQ ID NO: 36) | | |
| Variant 2 | CTCTCCGCACGACGGACGCTAATCTTACAAGGGCGTAGTGTATGTC | 15 | 22.7 |
| | GTGCG (SEQ ID NO: 37) | | |

Piperaquine

| | | | |
|---|---|---|---|
| Parent | CTCTCGGGACGACCACACGCTAGTTGCGCCGAATGGCCGTGAAAGT | 14 | 0.84 |
| | CGTCCC (SEQ ID NO: 38) | | |
| Variant 1 | GACTCGCGAGAGCCACACGCTAGTTGCGCCGAATGGCCGTGAAAGC | 15 | 1.88 |
| | TCTCGC (SEQ ID NO: 39) | | |
| Variant 2 | GACTCGGCAACGGCACACGCTAGTTGCGCCGAATGGCCGTGAAACC | 15 | 4.28 |
| | GTTGCC (SEQ ID NO: 40) | | |

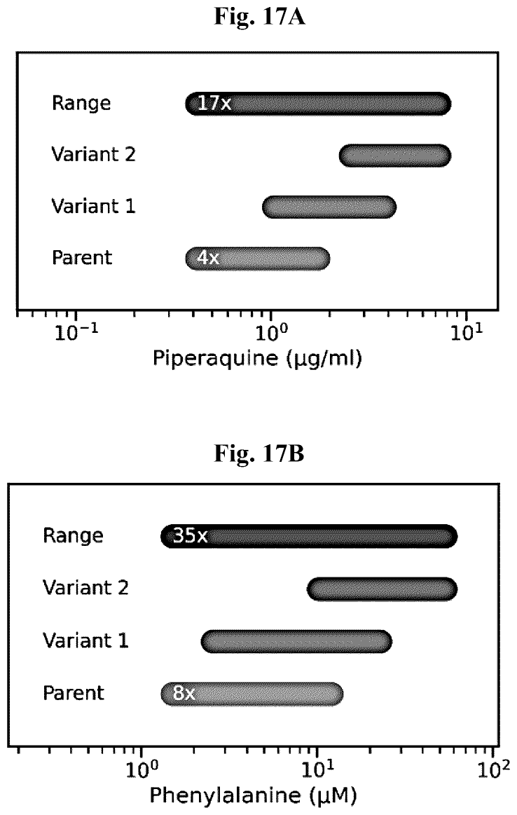

Surprisingly, changing the stem sequences resulted in SSAs with the same target specificity but altered response curves (FIGS. 16C and 16D). If we crudely define the linear range of each SSA as 20% to 80% activity, using this range of different stems allows us to take a single ligand-binding region and generate sensors with >4-fold greater dynamic range than the parent SSA (FIGS. 17A and 17B; Table 4). The results are very similar for both PQ and Phe suggesting that this is a generalizable finding. These data suggest that it is possible to greatly expand the dynamic range of a given SSA by tweaking the sequences in the structural elements as a general method.

TABLE 4
Dynamic range of SSAs of Example
15 using different cut-off values

| | | | | | | |
|---|---|---|---|---|---|---|
| | | | 20%-80% | | | 10%-90% |
| EC₅₀ | | | (fold- | | | (fold- |
| (μM) | 20% | 80% | change) | 10% | 90% | change) |

Phenylalanine

| | | | | | | | |
|---|---|---|---|---|---|---|---|
| Parent | 4.00 | 1.50 | 12.06 | 8.03 | 0.83 | 4.31 | 29.16 |
| Variant 1 | 7.66 | 2.55 | 22.82 | 8.96 | 1.34 | 43.11 | 32.24 |
| Variant 2 | 22.7 | 10.22 | 53.70 | 5.25 | 6.36 | 91.10 | 14.33 |
| Combined | NA | 1.50 | 53.70 | 35.74 | 0.83 | 91.10 | 109.26 |

Piperaquine

| | | | | | | | |
|---|---|---|---|---|---|---|---|
| Parent | 0.84 | 0.42 | 1.75 | 4.21 | 0.28 | 2.70 | 9.77 |
| Variant 1 | 1.88 | 1.03 | 3.84 | 2.86 | 0.73 | 5.86 | 7.98 |
| Variant 2 | 4.28 | 2.55 | 7.30 | 3.72 | 1.90 | 9.95 | 5.24 |
| Combined | NA | 0.42 | 7.30 | 17.55 | 0.28 | 9.95 | 35.96 |

### Example 16: Macromolecular Crowding Reagents can Greatly Increase Sensitivity of an SSA

The environment in a cell is very different to the environment in a tube containing an aqueous solution. One of the key differences is the presence of a large quantity of lipid-enclosed structures (the ER, the Golgi, vesicles, etc.) and a high density of proteins, often in large macromolecular complexes like the ribosome. The effect of these is to cause ‘macromolecular crowding’—small molecules like metabolites and drugs are at higher concentrations in the much-reduced aqueous space. Some of these effects can be mimicked in vitro by the addition of inert ‘macromolecular crowding reagents’ like polyethylene glycol (PEG) or Ficoll. Addition of these can greatly boost the kinetics of enzyme reactions and increase the rate of molecular interactions. We thus wished to test whether macromolecular crowding reagents could alter the dynamic range of an SSA.

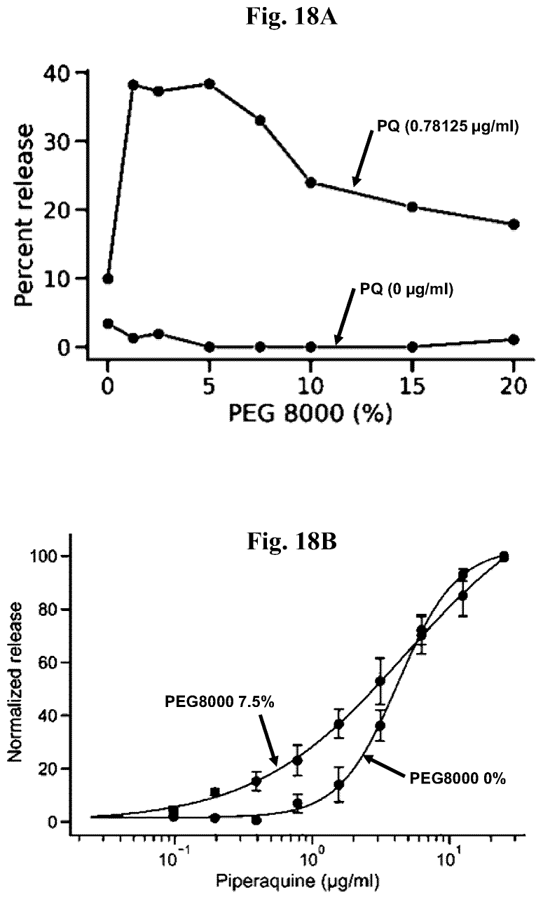

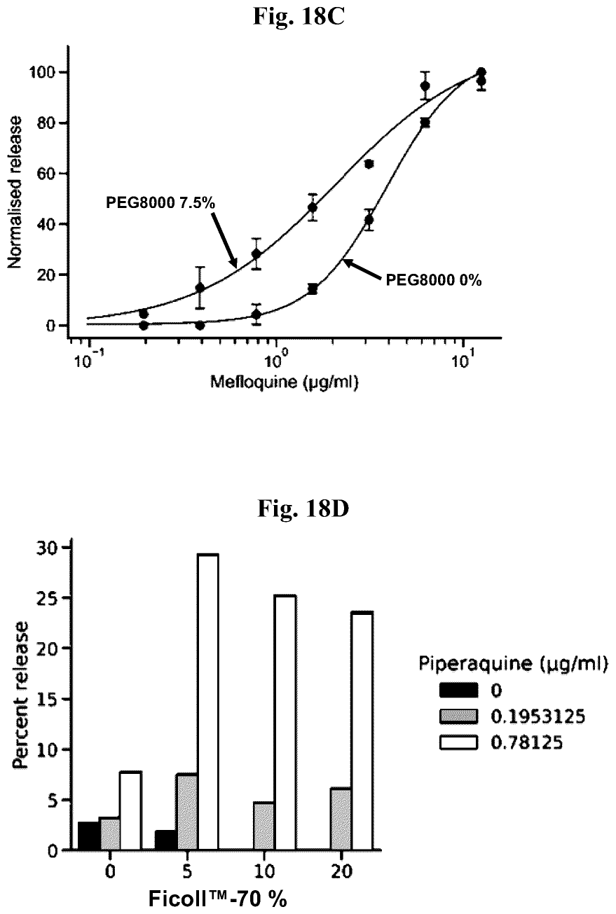

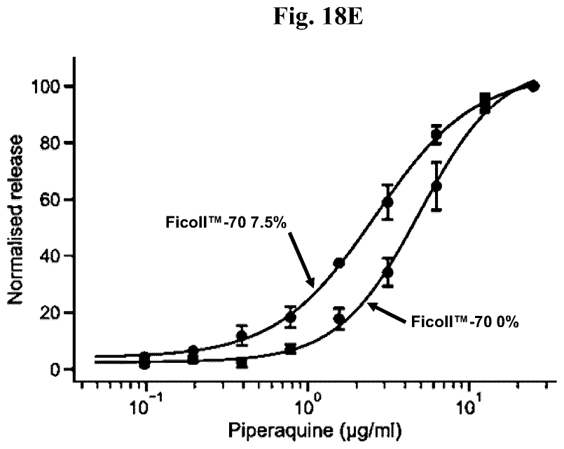

Addition of either PEG 8000 or Ficoll-70 caused a strong shift in the response curves of the PQ and mefloquine (MQ) SSAs (FIGS. 18A-18E). Addition of PEG or Ficoll extended the dynamic range of the PQ and MQ sensor by >2-fold (FIGS. 18B, 18C, and 18E). Strikingly, they also extend the sensitivity of each sensor at low concentrations (FIG. 18A and FIG. 18D) allowing detection of much lower target concentrations than in normal buffer. For the PQ sensor, the limit of detection (estimated here as 10% release) is improved 5.7-fold with PEG and 3.8-fold with Ficoll. For the MQ sensor, the limit of detection is improved 3.2-fold with PEG (FIG. 18C). These data suggest that macromolecular crowding reagents can greatly extend the dynamic range of SSAs and can greatly increase their sensitivity. We anticipate that this will be particularly useful for samples where target concentrations are extremely low such as single cell applications.

### Example 17: Barcode SSAs with an Immobilized Short Common Oligo and a Released LBO

In some embodiments of the barcode SSAs described here, the LBO is immobilized and the barcode is on the SRO which is released upon ligand binding. However, the present Example demonstrates that it is possible to reverse the architecture so that the SRO is now immobilized and the barcode is present on the LBO which is now released upon ligand binding (FIG. 19A). The same ligand-binding assay is then carried out and the barcodes can be read out from the LBOs that are released into the supernatant. To demonstrate that this works, we generated a barcode SSA based on a tyrosine aptamer from Yang et al., 2014 to this architecture. The SRO is immobilized and the barcoded LBO is released upon tyrosine binding. The barcode we used corresponds to the T7 promoter and we detect its release by using it to direct transcription of a Baby Spinach template (as described in previous Examples) though we note that the release could also be directly measured by sequencing (FIG. 19B). We show that this “flipped” architecture works to read out tyrosine-induced activation of the barcode SSA (FIG. 19C). Ultimately, we demonstrate herein that both architectures (i.e., ‘immobilized LBO+barcode SRO’ or ‘immobilized SRO+barcode LBO’) may be successfully employed to measure ligand levels using barcode readouts of ligand-induced dissociation of the SRO:LBO duplex into SRO and LBO monomers.

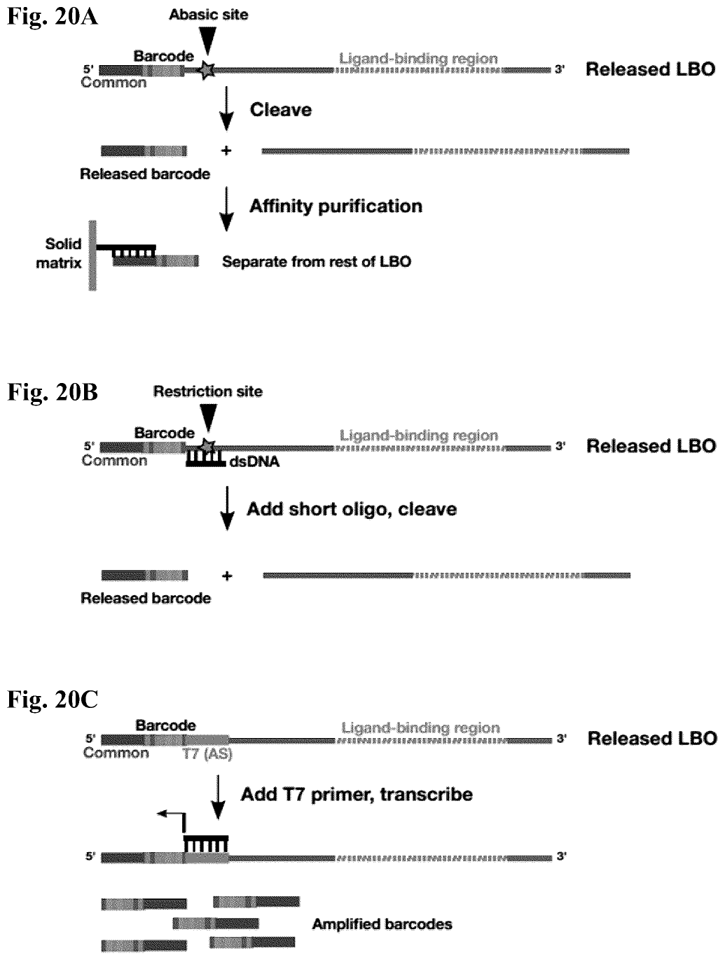

Finally, we note that in this reversed architecture illustrated in FIGS. 19A and 19B, it may not be necessary to sequence the entire barcode LBO to read the DNA barcode. Indeed, sequencing the entire barcode LBO could be problematic if the ligand binding region of the LBO included one or more unusual XNAs, mixtures of different XNAs, or any other molecule not recognizable by a DNA polymerase enzyme. In such as case, the DNA barcode could either be released from the LBO by enzymatic cleavage (FIGS. 20A and 20B) or transcribed from the LBO (FIG. 20C) or other known methods. Thus, these architectures do not require the reading of any of the bases in the ligand-binding region, allowing us to retain any chemistry in the ligand-binding region—including, but not limited to, XNAs and other non-standard bases. Again, it is enough to read the DNA barcode to know which ligand binding region recognized its ligand.

### Example 18: Summary

Structure-switching aptamers are powerful tools for detecting small molecules and other ligands of interest. Binding of a specific small molecule ligand drives a conformational change in an SSA. This is typically detected through a change in fluorescence, in electrical conductance, or spectroscopic properties. The downside of all such readouts is that they are identical for all SSAs—if multiple SSAs with the same readout are mixed into a single assay volume it is impossible to tell which of them has generated a detected signal and thus which has bound its ligand. Here, in some embodiments, we introduce a novel way to detect ligand binding by an SSA: the release of a barcoded short oligo. If each SSA in a mix has a different barcode, we can thus use sequencing to determine which SSAs have bound their ligand and released their barcode. We call these barcode SSAs and we have shown that: barcode release can be used to measure ligand binding to a barcode SSA; barcode release is specific to the correct ligand and that it requires the correct ligand binding region; either existing SSAs or other aptamers can be adapted to be barcode SSAs using the architecture we describe here; each barcode SSA behaves as an independent sensor in a mixture of barcode SSAs allowing the multiplexed detection of many hundreds of ligands in the same assay volume; and that a barcode SSA can detect its ligand and release its defined barcode even in the context of an extremely complex mixture of biomolecules. Furthermore, we set out a method to generate barcode SSAs which allows the correct matching of ligand binding oligo and barcode release oligo in bulk. This allows the efficient generation of large numbers of independent barcode SSA sensors without having to assemble them individually. We further describe how to increase the chemical (e.g., using XNAs) and physical (e.g., using spacers) space of the ligand binding region to increase the number of different molecules that can be detected. To facilitate correct pairing, we describe the synthesis of a unimolecular “pre-SSA” containing both the LBO and SRO, which is allowed to fold before being cleaved to generate a correctly hybridized LBO/SRO pairing. We also describe strategies for increasing the linear detection range for a given ligand of interest by incorporating multiple SSAs having LBOs with identical ligand binding regions but differing in their adjacent structural elements. Lastly, we demonstrate that the presence of macromolecular crowding reagents can greatly extend the sensitivity and/or dynamic range of SSAs, which may be particularly useful for samples where target concentrations are extremely low such as single cell applications.

Together we believe that we have shown that barcode SSAs allow the multiplexed detection of many different ligands in parallel in the context of a complex mixture and that we have efficient ways to generate such sensors. We believe that barcode SSAs can be applied to many different biosensor needs, including single cell metabolomics.

### REFERENCES

Coonahan, E. S., Yang, K. A., Pecic, S., De Vos, M., Wellems, T. E., Fay, M. P., Andersen, J. F., Taming, J., & Long, C. A. (2021). Structure-switching aptamer sensors for the specific detection of piperaquine and mefloquine. Science translational medicine, 13(585), eabe1535.
Conrad, T., Plumbom, I., Alcobendas, M. et al. Maximizing transcription of nucleic acids with efficient T7 promoters. Commun Biol 3, 439 (2020).
Okuda, M., Fourmy, D., & Yoshizawa, S. (2017). Use of Baby Spinach and Broccoli for imaging of structured cellular RNAs. Nucleic acids research, 45(3), 1404-1415.
Song, K. M., Jeong, E., Jeon, W., Cho, M., & Ban, C. (2012). Aptasensor for ampicillin using gold nanoparticle based dual fluorescence-colorimetric methods. Analytical and bioanalytical chemistry, 402(6), 2153-2161.
Warner, K. D., Chen, M. C., Song, W., Strack, R. L., Thorn, A., Jaffrey, S. R., & Ferré-D'Amaré, A. R. (2014). Structural basis for activity of highly efficient RNA mimics of green fluorescent protein. Nature structural & molecular biology, 21(8), 658-663.
Yang, K. A., Barbu, M., Halim, M., Pallavi, P., Kim, B., Kolpashchikov, D. M., Pecic, S., Taylor, S., Worgall, T. S., & Stojanovic, M. N. (2014). Recognition and sensing of low-epitope targets via ternary complexes with oligonucleotides and synthetic receptors. Nature chemistry, 6(11), 1003-1008.
Yang, K. A., Pei, R., & Stojanovic, M. N. (2016). In vitro selection and amplification protocols for isolation of aptameric sensors for small molecules. Methods (San Diego, Calif.), 106, 58-65.
Yang, K. A., Chun, H., Zhang, Y., Pecic, S., Nakatsuka, N., Andrews, A. M., Worgall, T. S., & Stojanovic, M. N. (2017). High-Affinity Nucleic-Acid-Based Receptors for Steroids. ACS chemical biology, 12(12), 3103-3112. https://doi.org/10.1021/acschembio.7b00634
US 2019/0242030 A1

1-34. (canceled)

35. A system for detecting one or more target ligands in a fluid sample, the system comprising one or more ligand-sensing complexes, each ligand-sensing complex being specific for a target ligand and comprising a ligand-binding oligonucleotide (LBO) hybridized to a corresponding short-release oligonucleotide (SRO) to form an LBO/SRO pairing, the LBO comprising a ligand-binding region that specifically binds to the target ligand and an SRO hybridization region sufficiently complementary to a corresponding LBO hybridization region comprised in the SRO to enable formation of the LBO/SRO pairing, wherein the SRO or LBO further comprises a barcode region informative with respect to the target ligand recognized by the ligand-sensing complex, and wherein binding of the target ligand to the ligand-binding region of the LBO drives a conformational change triggering dissociation of the LBO/SRO pairing, thereby enabling the released barcode region to be used as a readout for the presence or concentration of the target ligand in the fluid sample.

36. The system of claim 35, wherein:
(i) the LBO or SRO comprises an affinity tag or moiety;
(ii) the ligand-sensing complex is immobilized on a solid matrix via binding of the LBO to the matrix such that the LBO remains immobilized upon release of barcoded SRO following target ligand binding; or wherein the ligand-sensing complex is immobilized on a solid matrix via binding of the SRO to the matrix such that the SRO remains immobilized upon release of barcoded LBO following target ligand binding; or
(iii) both (i) and (ii).

37. The system of claim 35, which comprises a plurality of the same or different ligand-sensing complexes that are not physically or spatially separated or arranged, thereby reducing the volume of fluid sample required.

38. The system of claim 35, wherein:
(i) the system comprises a plurality of the same or different ligand-sensing complexes each having similar LBO/SRO hybridization characteristics;
(ii) the system comprises a plurality of the same or different ligand-sensing complexes each having LBO/SRO pairing melting Temperatures™ that do not differ by more than 10, 9, 8, 7, 6, 5, 4, 3, 2, or degrees;
(iii) the LBO and SRO hybridization regions of each of the ligand-sensing complexes in the system have the same nucleotide composition and/or the same nucleotide sequence; or
(iv) any combination of (i) to (iii).

39. The system of claim 35, wherein the LBO and the SRO of each ligand-sensing complex comprises a terminal end resulting from cleavage at an abasic site, a photocleavable site, or an enzymatic cleavage site, of a unimolecular polynucleotide comprising the nucleotide sequences of both the LBO and the SRO.

40. The system of claim 35, which is a multiplexed system comprising a plurality of different ligand-sensing complexes, each complex being designed to release a different barcode upon binding of a different target ligand, thereby enabling detection of a plurality of different target ligands from a single fluid sample volume.

41. The system of claim 40, wherein a plurality of different ligand-sensing complexes is heterogeneously hybridized to the same surface in a location agnostic fashion such that the particular locations of the complexes on the surface are uncontrolled or not readily determinable.

42. The system of claim 35, wherein the ligand-binding region of the LBO comprises or is derived from an aptamer or a structure-switching aptamer.

43. The system of claim 35, wherein the system comprises multiple species of ligand-sensing complexes that bind to the same target ligand, wherein each species comprises LBOs having identical ligand-binding regions but that differ in their non-ligand binding structural elements, thereby providing a plurality of LBO species that bind to the target ligand at different but overlapping dynamic ranges such that the overall dynamic range of the system is greater than that of a single LBO species.

44. The system of claim 35, wherein:
(i) the LBO species comprise aptamers having stem-loop structures, and wherein each of the LBO species differ in their stem structures,
(ii) the LBO and/or SRO is/are at least partly composed of naturally and/or artificially produced DNA, RNA, XNA, peptides, peptoids, lipids, polysaccharides, or any combination thereof;
(iii) the barcode region is constructed or adapted to facilitate capture, amplification, and/or sequencing;
(iv) the system further comprises an inert macromolecular crowding agent for admixture with the fluid sample at a concentration sufficient to increase the ligand-sensing complexes' sensitivity and/or dynamic range with respect to its target ligand, as compared to in the absence of the inert macromolecular crowding agent;
(v) the fluid sample is a biological sample;
(vi) the one or more target ligands comprise a small molecule, protein, peptide, amino acid, antigen, fatty acid, monosaccharide, disaccharide, oligosaccharide, polysaccharide, metabolite, cytokine, chemokine, drug or drug metabolite, or any combination thereof; or
(vii) any combination of (i) to (vi).

45. A method for detecting or measuring a ligand in a fluid sample, the method comprising:
(a) providing the system as defined in claim 35;
(b) contacting the system with the fluid sample; and
(c) detecting the released barcoded SRO(s) as a readout for the presence or concentration of each of the target ligand(s) in the fluid sample.

46. The method of claim 45, wherein (c) comprises: capturing the released barcoded SRO; amplifying the barcode region of the released SRO; sequencing the barcode region of the released SRO; or any combination thereof.

47. A method for preparing one or more ligand-sensing complexes as defined in claim 35, the method comprising:
(a) providing one or more unimolecular polynucleotides, each unimolecular polynucleotide comprising the nucleotide sequences of both the LBO and the SRO separated by a cleavage site positioned therebetween;
(b) allowing the complementary LBO and SRO hybridization regions to hybridize; and
(c) cleaving the polynucleotide at the cleavage site, thereby producing the one or more ligand-sensing complexes having separate LBO and SRO molecules that are correctly paired.

48. The method of claim 47, wherein:
(i) the one or more unimolecular polynucleotides are immobilized on a solid matrix prior to or following the cleavage in (c) such that only one of the LBO and SRO remains immobilized following cleavage;
(ii) the cleavage in (c) comprises cleaving a mixture of different unimolecular polynucleotides in the same reaction solution, thereby producing a plurality of different ligand-sensing complexes in parallel; or
(iii) both (i) and (ii).

49. A unimolecular polynucleotide that is precursor of a ligand-sensing complex as defined in claim 35, the unimolecular polynucleotide comprising a ligand-binding oligonucleotide sequence (LBO) and a short-release oligonucleotide sequence (SRO), the LBO comprising a ligand-binding region that specifically binds to a target ligand and an SRO hybridization region sufficiently complementary to a corresponding LBO hybridization region comprised in the SRO to enable formation of the LBO/SRO pairing, wherein the SRO or LBO further comprises a barcode region informative with respect to the target ligand recognized by the ligand-sensing complex, and wherein binding of the target ligand to the ligand-binding region of the LBO drives a conformational change triggering dissociation of the LBO/SRO pairing, thereby enabling the released barcode region to be used as a readout for the presence or concentration of the target ligand in the fluid sample.

50. An aptamer-based detection system having improved dynamic range for a target ligand, the system comprising a plurality of ligand-binding oligonucleotide (LBO) species that bind to the same target ligand, wherein the plurality of LBO species comprises:
(a) LBO species having different ligand-binding regions that specifically bind to the target ligand but with different affinities;
(b) LBO species having identical ligand-binding regions that specifically bind to the target ligand but that differ in their non-ligand binding structural elements; or
(c) both (a) and (b),
thereby producing a plurality of LBO species that bind to the target ligand at different but overlapping dynamic ranges such that the overall dynamic range of the system is greater than that of a single LBO species.

51. The aptamer-based detection system of claim 50, wherein:
(i) the LBO species comprise structure-switching aptamers;
(ii) the LBO species comprise aptamers having stem-loop structures, and wherein each of the LBO species in (b) differ in their stem structures; or
(iii) both (i) and (ii).

52. A method for increasing the sensitivity and/or dynamic range of an aptamer for its ligand, the method comprising: providing a sample comprising or suspected of comprising a ligand of interest; contacting the sample with an aptamer that binds to the ligand of interest in the presence of a concentration of an inert macromolecular crowding agent sufficient to increase the aptamer's sensitivity and/or dynamic range with respect to its ligand, as compared to the aptamer's sensitivity and/or dynamic range in a corresponding sample lacking the inert macromolecular crowding agent.

53. The method of claim 52, wherein:
(i) the macromolecular crowding agent is or comprises one or more of: polyethylene glycol; a neutral branched hydrophilic polysaccharide; dextran; a protein; or other inert macromolecule;
(ii) the concentration of the macromolecular crowding agent is at least 0.5, 0.6, 0.7, 0.8, 0.9, 1, 1.5, 2, 2.5, 3, 3.5, 4, 4.5, or 5% to about 6, 6.5, 7, 7.5, 8, 8.5, 9, 9.5, 10, 11, 12, 13, 14, 15, 16, 18, 19, 20, 21, 22, 23, 24, 25, 26, 27, 28, 29, 30, 35, 40, 45, 50, 55, 60, 65, or 70% (w/v)
(iii) both (i) and (ii).

54. The method of claim 52, wherein the aptamer is a structure-switching aptamer.


SEQUENCE LISTING (49 sequences)

SEQ ID NO: 1 - DNA, 73 bp, synthetic construct
  source 1..73: mol_type = other DNA
ggtgctcacactctactcaacagtagcgaactactggacccgtccttcacctatagtgagtcgtattagactc

SEQ ID NO: 2 - DNA, 51 bp, synthetic construct
  source 1..51: mol_type = other DNA
ctctcgggacgacggacgctaatcttacaagggcgtagtgtatgtcgtccc

SEQ ID NO: 3 - DNA, 51 bp, synthetic construct
  source 1..51: mol_type = other DNA
ctctcgggacgacaggagacttagatgttatatcaggcgtcgcgtcgtccc

SEQ ID NO: 4 - DNA, 38 bp, synthetic construct
  source 1..38: mol_type = other DNA
ctctcgggacgacggcccgatctcagagtagtcgtccc

SEQ ID NO: 5 - DNA, 32 bp, synthetic construct
  source 1..32: mol_type = other DNA
gtcgtcccgagagtaatacgactcactatagg

SEQ ID NO: 6 - DNA, 32 bp, synthetic construct
  source 1..32: mol_type = other DNA
gtcgtcccgagagaattaaccctcactaaagg

SEQ ID NO: 7 - DNA, 58 bp, synthetic construct
  source 1..58: mol_type = other DNA
taagatgactcggcaacggcacacgctagttgcgccgaatggccgtgaaaccgttgcc

SEQ ID NO: 8 - DNA, 34 bp, synthetic construct
  source 1..34: mol_type = other DNA
tgccgttgccgagtctaatacgactcactatagg

SEQ ID NO: 9 - DNA, 34 bp, synthetic construct
  source 1..34: mol_type = other DNA
tgccgttgccgagtcaattaaccctcactaaagg

SEQ ID NO: 10 - DNA, 57 bp, synthetic construct
  source 1..57: mol_type = other DNA
taagatctctcgggacgacggcagtctataccccgtatcgccgaaggttgtcgtccc

SEQ ID NO: 11 - DNA, 34 bp, synthetic construct
  source 1..34: mol_type = other DNA
ccgtcgtcccgagagtaatacgactcactatagg

SEQ ID NO: 12 - DNA, 34 bp, synthetic construct
  source 1..34: mol_type = other DNA
ccgtcgtcccgagagaattaaccctcactaaagg

SEQ ID NO: 13 - DNA, 40 bp, synthetic construct
  source 1..40: mol_type = other DNA
ctctcgggacgacgcgggcggttgtatagcgggtcgtccc

SEQ ID NO: 14 - DNA, 46 bp, synthetic construct
  source 1..46: mol_type = other DNA
taagatgactcgcgagagcgcgggcggttgtatagcgggctctcgc

SEQ ID NO: 15 - DNA, 46 bp, synthetic construct
  source 1..46: mol_type = other DNA
taagatgactcggcaacgggcgggcggttgtatagcggccgttgcc

SEQ ID NO: 16 - DNA, 32 bp, synthetic construct
  source 1..32: mol_type = other DNA
gctctcgcgagtctaatacgactcactatagg

SEQ ID NO: 17 - DNA, 32 bp, synthetic construct
  source 1..32: mol_type = other DNA
ccgttgccgagtctaatacgactcactatagg

SEQ ID NO: 18 - DNA, 83 bp, synthetic construct
  source 1..83: mol_type = other DNA
ctctcgggacgaccgcggtagtcttaacctaaagcggtgtcaggtcgtcccgtcgtcccgagagaattaaccctcactaaagg

SEQ ID NO: 19 - DNA, 83 bp, synthetic construct
  source 1..83: mol_type = other DNA
ctctcgggacgactatgccggagtagcggatgccaccagttatgtcgtcccgtcgtcccgagagaattaaccctcactaaagg

SEQ ID NO: 20 - DNA, 40 bp, synthetic construct
  source 1..40: mol_type = other DNA
ctctcgggacgacggcccgaaaaaaaaaagtagtcgtccc

SEQ ID NO: 21 - DNA, 40 bp, synthetic construct
  source 1..40: mol_type = other DNA
ctctcgggacgacggcccgtttttttttagtagtcgtccc

SEQ ID NO: 22 - RNA, 50 nt, synthetic construct
  source 1..50: mol_type = other RNA
ggtgaaggacgggtccagtagttcgctactgtgagtagagtgtgagcacc

SEQ ID NO: 23 - DNA, 55 bp, synthetic construct
  source 1..55: mol_type = other DNA
gtcgtcccgagaggtcgcctataagtatttccctatagtgagtcgtattaatttc

SEQ ID NO: 24 - DNA, 24 bp, synthetic construct
  source 1..24: mol_type = other DNA
gaaattaatacgactcactatagg

SEQ ID NO: 25 - DNA, 18 bp, synthetic construct
  source 1..18: mol_type = other DNA
tatgactcgggcagtgca

SEQ ID NO: 26 - DNA, 23 bp, synthetic construct
  source 1..23: mol_type = other DNA
cactgccctgcactgcccgagtc

SEQ ID NO: 27 - DNA, 20 bp, synthetic construct
  source 1..20: mol_type = other DNA
atattttttgggtttttatt

SEQ ID NO: 28 - DNA, 20 bp, synthetic construct
  source 1..20: mol_type = other DNA
tatgttttggggtttttata

SEQ ID NO: 29 - DNA, 20 bp, synthetic construct
  source 1..20: mol_type = other DNA
tattttttggggtttttata

SEQ ID NO: 30 - DNA, 20 bp, synthetic construct
  source 1..20: mol_type = other DNA
caggttttggtttttttgtg

SEQ ID NO: 31 - DNA, 20 bp, synthetic construct
  source 1..20: mol_type = other DNA
caggttttgtttttttggtg

SEQ ID NO: 32 - DNA, 20 bp, synthetic construct
  source 1..20: mol_type = other DNA
caggttttgttttttttgtg

SEQ ID NO: 33 - DNA, 20 bp, synthetic construct
  source 1..20: mol_type = other DNA
cagtttttggtttttttgtg

SEQ ID NO: 34 - DNA, 20 bp, synthetic construct
  source 1..20: mol_type = other DNA
gttgttttattttttttatt

SEQ ID NO: 35 - DNA, 20 bp, synthetic construct
  source 1..20: mol_type = other DNA
gtttttttaatttttttatt

SEQ ID NO: 36 - DNA, 51 bp, synthetic construct
  source 1..51: mol_type = other DNA
ctctcgggcagacggacgctaatcttacaagggcgtagtgtatgtctgccc

SEQ ID NO: 37 - DNA, 51 bp, synthetic construct
  source 1..51: mol_type = other DNA
ctctccgcacgacggacgctaatcttacaagggcgtagtgtatgtcgtgcg

SEQ ID NO: 38 - DNA, 52 bp, synthetic construct
  source 1..52: mol_type = other DNA
ctctcgggacgaccacacgctagttgcgccgaatggccgtgaaagtcgtccc

SEQ ID NO: 39 - DNA, 52 bp, synthetic construct
  source 1..52: mol_type = other DNA
gactcgcgagagccacacgctagttgcgccgaatggccgtgaaagctctcgc

SEQ ID NO: 40 - DNA, 52 bp, synthetic construct
  source 1..52: mol_type = other DNA
gactcggcaacggcacacgctagttgcgccgaatggccgtgaaaccgttgcc

SEQ ID NO: 41 - DNA, 57 bp, synthetic construct
  source 1..57: mol_type = other DNA
taagatctctcgggacgacgcccgcatgttccatggatagtcttgactagtcgtccc

SEQ ID NO: 42 - DNA, 16 bp, synthetic construct
  source 1..16: mol_type = other DNA
gccgttgccgagtcta

SEQ ID NO: 43 - DNA, 15 bp, synthetic construct
  source 1..15: mol_type = other DNA
gtcgtcccgagagta

SEQ ID NO: 44 - DNA, 58 bp, synthetic construct
  source 1..58: mol_type = other DNA
taagatgactcgggcagtgcacacgctagttgcgccgaatggccgtgaaacactgccc

SEQ ID NO: 45 - DNA, 34 bp, synthetic construct
  source 1..34: mol_type = other DNA
tgcactgcccgagtctaatacgactcactatagg

SEQ ID NO: 46 - DNA, 51 bp, synthetic construct
  source 1..51: mol_type = other DNA
taagatggctctcgggacgactatgttttggggtttttatagtcgtcccga

SEQ ID NO: 47 - DNA, 51 bp, synthetic construct
  source 1..51: mol_type = other DNA
taagatggctctcgggacgaccagtttttggtttttttgtggtcgtcccga

SEQ ID NO: 48 - DNA, 43 bp, synthetic construct
  source 1..43: mol_type = other DNA
taagatggctctcgggacgacgtttaatttattgtcgtcccga

SEQ ID NO: 49 - DNA, 15 bp, synthetic construct
  source 1..15: mol_type = other DNA
gtcgtcccgagagcc